(19) United States
(12) Patent Application Publication
(10) Pub. No.: US 2025/0177482 A1
(43) Pub. Date: Jun. 5, 2025

(54) COMPLEMENT FACTOR I DOSING REGIMENS FOR TREATING OCULAR DISEASES

(71) Applicant: Vertex Pharmaceuticals Incorporated, Boston, MA (US)

(72) Inventors: Grant E. BLOUSE, Burlingame, CA (US); Natacha LE MOAN, South San Francisco, CA (US); Tom KNUDSEN, Burlingame, CA (US)

(21) Appl. No.: 18/935,289

(22) Filed: Nov. 1, 2024

Related U.S. Application Data
(63) Continuation of application No. PCT/US2023/066620, filed on May 4, 2023.
(60) Provisional application No. 63/338,392, filed on May 4, 2022.

(51) Int. Cl. A61K 38/17; A61K 9/00; A61K 38/38

(52) U.S. Cl. CPC A61K 38/1725; A61K 9/0048; A61K 38/177; A61K 38/385

(57) ABSTRACT

Provided herein are dosing regimens for the treatment of ocular conditions such as age-related macular degeneration (AMD). The regimens comprise administering to a subject in need thereof fusion constructs comprising a Complement Factor I (CFI) and a binding partner at specified doses and frequencies.

### CROSS REFERENCE TO RELATED APPLICATION

This application is a continuation of International PCT Patent Application No. PCT/US2023/066620, filed on May 4, 2023, which claims priority to U.S. Provisional Application No. 63/338,392, filed on May 4, 2022, the contents of which are incorporated herein by reference in their entireties.

### REFERENCE TO AN ELECTRONIC SEQUENCE LISTING

The contents of the electronic sequence listing (VTEX_708_01US_SeqList_ST26.xml; Size: 18,753 bytes; and Date of Creation: Oct. 29, 2024) are herein incorporated by reference in its entirety.

### BACKGROUND

The complement system includes the classical, lectin and alternative pathways, and is tightly controlled by a number of regulators. Complement Factor I (CFI) is one such regulator, and acts to regulate the complement system by cleaving C4b and C3b proteins, thereby inactivating these proteins. Such cleavage results in inhibition of the classical, lectin and alternative pathways, respectively, thus ultimately preventing the assembly of the C3 and C5 convertase enzymes. CFI is encoded as a proenzyme and is then activated by proteolytic cleavage into a heterodimeric glycoprotein having a heavy chain (A chain) and a light chain (B chain) that are connected by a disulfide linkage. CFI is processed into its active form post-translationally by furin to generate the two-chain mature protein.

Dysregulated CFI, mutated and dysfunctional CFI (i.e. genetic variants), or CFI deficiency have been implicated in diseases involving the complement system. Dysregulation or excess activation of the complement system has been associated with ocular diseases such as age-related macular degeneration (AMD). The progression of AMD leads to the damage of the macula, atrophy of the retinal pigment epithelium (RPE), and loss of photoreceptors causing blurred and eventual loss of vision. Carrying rare genetic variants of CFI and/or low CFI levels have been associated with AMD and the progression of the disease. According to the National Eye Institute of the National Institutes of Health (NIH), AMD is a leading cause of vision loss for adults, with more than 190 million people affected globally in 2020. There are two types of AMD: wet AMD and dry AMD. Wet AMD is less common, and is characterized by abnormal growth of blood vessels in the back of the eye, damaging the macula. Dry AMD is characterized by the thinning of the macula, and can progress to geographic atrophy (GA), which is characterized by progressive and irreversible loss of retinal tissue. GA is a leading cause of blindness, and affects over 5 million people globally. Methods are needed for modulating or inhibiting the complement system to maintain the balance of regulation for the treatment of ocular diseases such as AMD and GA. Provided herein are compositions and methods to address the dysfunction and/or dysregulation in the complement system.

### SUMMARY

In one aspect, provided herein is a method of treating or preventing an ocular condition, in a human subject in need thereof, comprising administering to the subject a fusion construct comprising a Complement Factor I comprising the amino acid sequence of SEQ ID NO. 5, or a variant thereof, and a binding partner, wherein the route of administration is intraocular, and wherein the fusion construct is administered at a dose of about 0.1 to about 10 mg per eye and/or administered at a frequency of about every 1 to 4 months. In some embodiments, the binding partner is serum albumin comprising the amino acid sequence of SEQ ID NO. 7, or a variant thereof. In some embodiments, the variant of CFI has at least 70%, at least 80%, at least 90%, or at least 95% identity with the amino acid sequence of SEQ ID NO.5. In some embodiments the variant of CFI comprises a CFI of SEQ ID NO: 5 comprising one or more of the substitutions of T377G, N422K, E457G, and N531G, comprises a CFI of SEQ ID NO: 5 comprising the substitutions of N422K, E457G, and N531G, or comprises a CFI of SEQ ID NO: 5 comprising the substitutions of T377G, E457G, and N531G. In some embodiments, the binding partner is a cofactor of CFI, for example Factor H (FH) or Complement Receptor 1 (CR1). In some embodiments, the Factor H binding partner comprises the amino acid sequence of SEQ ID NO. 22 or SEQ ID NO: 23, a domain thereof, or a part of a domain thereof. In some embodiments, the binding partner comprises a CR1, a domain thereof, or a part of a domain thereof. A CFI-FH fusion protein can further comprise a serum albumin, e.g. a serum albumin comprising the amino acid sequence of SEQ ID NO. 7, or a variant thereof. A CFI-CR1 fusion protein can further comprise a serum albumin, e.g. a serum albumin comprising the amino acid sequence of SEQ ID NO. 7, or a variant thereof. In some embodiments, the route of administration is intravitreal. In some embodiments, the ocular condition is dry Age-related Macular Degeneration (AMD). In some embodiments, the ocular condition is wet Age-related Macular Degeneration (AMD). In some embodiments, the ocular condition is Geographic Atrophy (GA). In some embodiments, the subject exhibits GA. In some embodiments, the human subject carries one or more mutations in the CFI gene selected from the group consisting of: G119R, L131R, V152M, G162D, R187Y, R187T, T203I, A240G, A258T, G287R, A300T, R317W, R339Q, V412M, P553S, K441R, R339Ter, R317Q, G261D, R187Q, and R187Ter. In some embodiments, the ocular condition is caused by one or more mutations to the CFI gene in the subject selected from the group consisting of: Phe13Val, Val20Ile, His40Arg, Cys43Phe, Asp44Asn, Pro50Ala, Cys54Arg, Cys54*, Ile55Phe, Pro64Leu, Asn70Thr, Thr72Ser, Phe82Ser, Pro83Gln, Ser90Asn, Cys106Arg, Thr107Ala, Glu109Ala, His118Arg, Gly119Arg, Gly125Arg, Val127Ala, Val129Gly, Leu131Arg, Met138Val, Met138Ile, Trp145*, Asn151Ser, Val152Met, Gly162Asp, Asp164Val, Asn177Ile, His183Arg, Val184Met, Arg187*, Arg187Gln, Gly188Ala, Phe198Leu, Arg202Ile, Thr203Ile, Tyr206Asn, Gln217His, Ser221Tyr, Asp224Asn, Cys229Arg, Val230Met, Val230Glu, Ala240Gly, Cys247Gly, Gly248Glu, Asp249Glu, Ala258Thr, Gly261Ser, Gly261Asp, Gly263Val, Lys267Asn, Gly280Asp, Gly287Arg, Glu290Asp, Ala300Thr, Glu303Lys, Glu305*, Ile306Val, Ile306Ser, Asp310Glu, Arg317Trp, Arg317Gln, Cys327Arg, Gly328Arg, Arg339*, Arg339Gln, Ile340Thr, Gly342Glu, Arg345Gln, Gly349Arg, Val355Met, Ala356Pro, Ile357Met, Gly362Ala, Tyr369Ser, Trp374Cys, Arg389His, Trp399Arg, Pro402Ser, Asp403Asn, Arg406Cys, Arg406His, Tyr411*, Val412Met, Ile416Leu, His418Leu, Gly424Asp, Asp429Glu, Ala431Thr, Ile433Val, Ile433Thr, Lys441Arg, Arg448Cys, Ala452Ser, Trp456Leu, Tyr459Ser, Gln462His, Cys467Arg, Arg474*, Arg474Gln, Asp477His, Gly487Cys, Ile492Leu, Gly500Arg, Arg502Cys, Gly512Ser, Asp519Asn, Lys522Thr, Asp524Val, Met532Val, Asn536Lys, Trp541*, Gly542Ser, Val543Ala, Trp546*, Glu548Gln, Pro553Ser, Glu554Val, Ser570Thr, Ile578Thr, and Gln580*. In some embodiments, the dose is about 0.1 mg to about 10 mg per eye. In some embodiments, the dose is about 0.1, 0.5, 1, 5, or 10 mg per eye. In some embodiments, the frequency is about every 1 month. In some embodiments, the frequency is about every 2, 3, or 4 months. In some embodiments, the fusion construct is administered to one eye. In some embodiments, the fusion construct is administered to both eyes sequentially. In some embodiments, the fusion construct is administered to both eyes simultaneously. In some embodiments, the subject's intraocular pressure is stable after treatment. In some embodiments, the subject's retinal thickness is stable after treatment. In some embodiments, the fusion construct exhibits an activity in the subject that is at least equivalent to the activity that a plasma-derived or naturally-occurring CFI protein exhibits.

### BRIEF DESCRIPTION OF THE DRAWINGS

FIG. 1 is a drawing that depicts a model of an exemplary CFI-albumin (e.g. serum albumin, e.g. human serum albumin (HSA)) fusion construct comprising serum albumin fused with CFI, wherein the CFI comprises a wild type CFI.

FIG. 2A is a graph showing the amount of C3a detected in samples from the vitreous humor following an intravitreal injection (IVT) of a fusion construct comprising wild type CFI and a human serum albumin (CFI-HSA), at a dose of 250 μg or 500 μg in an African Green Monkeys (AGM) primate model.

FIG. 2B is a plot showing the ratio of C3b/iC3b detected in samples from the vitreous humor following an intravitreal injection (IVT) of a fusion construct comprising wild type CFI and a human serum albumin (CFI-HSA), at a dose of 1.0 mg in an AGM primate model.

FIG. 3A is a graph that depicts vitreous pharmacokinetics (PK) in AGM. The construct comprising CFI-HSA was injected intravitreally at 0.25 mg per eye or 1 mg per eye. The amount of the injected construct was measured over time. FIG. 3B is a graph that depicts vitreous PK modeling in humans based on the PK measured for AGM.

FIG. 4A are graphs that depict the clinical score via slit lamp for intravitreal injections of CFI-HSA in nonhuman primates. FIG. 4B is a graph that depicts the intraocular pressure measured over time in nonhuman primates post intravitreal injection of CFI-HSA. FIG. 4C is a graph that depicts the retinal thickness measured over time in nonhuman primates post intravitreal injection of CFI-HSA.

### DETAILED DESCRIPTION

The disclosure provides dosage regimens for fusion constructs comprising CFI and a binding partner, including for example, HSA, Factor H, or CR1, useful for modulating the signaling and amplification of the complement system in the ocular system. The disclosure also provides methods of making and using these fusion constructs, for example in treating an ocular disease or condition associated with complement dysregulation, such as dry age-related macular degeneration (AMD), wet AMD, and Geographic Atrophy (GA).

### I. Complement Factor I Fusion Constructs

Provided herein are Complement Factor I (CFI) fusion constructs, such fusion constructs comprising:
(a) a wild type CFI, or a variant thereof, and
(b) a binding partner.
   In some embodiments the binding partner comprises a serum albumin or a variant thereof. In some embodiments, the binding partner comprises a cofactor of CFI, for example Factor H, or CR1.

In some embodiments, the fusion constructs comprise a:
(a) Complement Factor I comprising the amino acid sequence of SEQ ID NO. 5, or a variant thereof, and
(b) a serum albumin comprising the amino acid sequence of SEQ ID NO. 7, or a variant thereof.

In some embodiments, the fusion constructs comprise a:
(a) Complement Factor I comprising the amino acid sequence of SEQ ID NO. 5, or a variant thereof,
(b) a Factor H cofactor comprising the amino acid sequence of SEQ ID NO. 22 or 23, or a domain thereof, and
(c) optionally a serum albumin comprising the amino acid sequence of SEQ ID NO. 7, or a variant thereof.

In some embodiments, the fusion constructs comprise a:
(a) Complement Factor I comprising the amino acid sequence of SEQ ID NO. 5, or a variant thereof,
(b) a CR1 cofactor, or a domain thereof, and
(c) optionally a serum albumin comprising the amino acid sequence of SEQ ID NO. 7, or a variant thereof.

As used herein, a wild type CFI refers to any naturally occurring full-length or nearly full-length CFI that is not a disease-causing CFI, with or without a signal sequence, and which may be of any species.

In some embodiments, a wild type CFI is plasma-derived. In some embodiments, a wild type CFI is a human wild type CFI. In some embodiments, a wild type, human CFI having a signal sequence comprises the amino acid sequence set forth in SEQ ID NO: 1 (as shown in Table 1 below). In some embodiments, a wild type CFI is a human CFI. In some embodiments, a wild type, human CFI does not include a signal sequence. In some embodiments, a wild type CFI without a signal sequence comprises the amino acid sequence set forth in SEQ ID NO: 5 (as shown in Table 1 below).

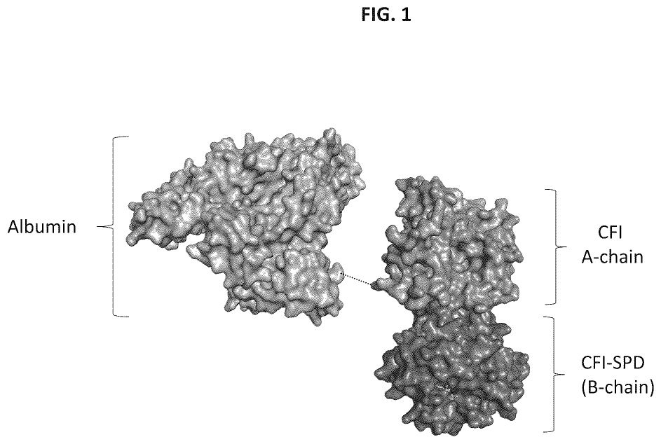

A wild type CFI comprises a heavy chain and a light chain, which are also referred to as the A-chain and B-chain, respectively. FIG. 1 depicts a schematic diagram of CFI showing the two chains.

Provided in Table 1 are exemplary base molecules used as components of the CFI fusion constructs. The base molecules of Table 1 were used to generate the CFI fusion constructs disclosed herein. The base molecules of Table 1 may be further part of a fusion construct, further described below.

Provided herein are fusion constructs that comprise variants to CFI, comprising one or more modifications with respect to a wild type CFI, referred to herein as “CFI variants.” As used herein, a “modification” to a wild type CFI includes: a deletion of one or more amino acid residues, a deletion of one or more domains, a substitution of one or more amino acid residues, an insertion (i.e. addition) of one or more amino acid residues, an insertion (i.e. addition) of one or more domains, an inversion of or one or more domains, and a substitution of one or more domains.

Variants of CFI may include those variants that impart an additional activity of the CFI on one or more of the classical, lectin and alternative pathways. In some embodiments, the modification with respect to a wild type CFI comprises any one or more of a deletion of one or more amino acid residues, a deletion of one or more CFI domains, a substitution of one or more amino acid residues, an insertion of one or more amino acid residues, an insertion of one or more CFI domains, and a swapping of one or more CFI domains. PCT/US2021/037278 and PCT/US2022/082179 provide variants of CFI, and are incorporated herein by reference in their entireties.

In some embodiments, the CFI variant comprises any one or more domains of CFI selected from: the serine protease domain (SPD), the Factor I membrane attack complex (FIMAC) domain, the SRCR domain, the low density lipoprotein receptor 1 (LDLr1) domain, and the low density lipoprotein receptor 2 (LDLr2) domain.

In some embodiments, the CFI variant comprises at least one modification corresponding to a wild type human CFI. In some embodiments, the CFI variant comprises at least one modification corresponding to a wild type non-human CFI. In some embodiments, the CFI variant comprises at least one modification corresponding to a wild type CFI having the amino acid sequence set forth in SEQ ID NO: 1 or SEQ ID NO: 5.

In some embodiments, the CFI variant is a chimera comprising one or more domains from a human CFI, and wherein the human CFI further comprises a substitution of one or more amino acid residues for amino acid residues of a corresponding region from a non-human species CFI. In some embodiments, the non-human species is mouse. In some embodiments, the CFI variant is a chimera, and wherein the modification comprises the substitution of one or more amino acid residues of the CFI with amino acid residues from a corresponding region of a non-CFI serine protease. In some embodiments, the non-CFI serine protease is trypsin.

In some embodiments, the CFI variant comprises an A chain and a B chain, wherein the CFI variant comprises one or more modifications at the interface of the A chain and the B chain.

In some embodiments, the CFI variant comprises a modification at any one or more positions corresponding to positions K14, Y20, D26, F29, R35, E38, M220, K221, S250, L304, P305, K306, L307, and S308 in a CFI having the amino acid sequence set forth in SEQ ID NO: 5. In some embodiments, the CFI variant comprises one or more of the substitutions selected from K14A, Y20A, Y20F, D26A, F29A, R35A, E38A, M220A, K221Q, S250A, S250L, L304G, P305G, K306G, L307G, and S308G, wherein the positions correspond to positions in a CFI having the amino acid sequence set forth in SEQ ID NO: 5. In some embodiments, the CFI variant comprises one or more of the combination of substitutions M220A; K221Q, and L304G; P305G; K306G; L307G; S308G, wherein the positions correspond to positions in a CFI having the amino acid sequence set forth in SEQ ID NO: 5.

In some embodiments, the CFI variant comprises one or more modifications at a C-terminal region of the CFI variant. In some embodiments, the CFI variant comprises a modification at any one or more positions corresponding to positions T377, W381, P384, Y403, A405, G406, Y408, Q409, D425, G556, R557, P558, P559, I560, and Y563 in a CFI having the amino acid sequence set forth in SEQ ID NO: 5. In some embodiments, the CFI variant comprises one or more of the substitutions selected from T377G, W381A, P384A, P384G, Y403F, A405S, G406R, G406A, Y408L, Q409D, Q409H, D425A, D425K, D425R, G556A, G556S, R557A, R557K, P558G, P558L, P558S, F559L, I560V, and Y563H, and/or a deletion of P384, wherein the positions correspond to positions in a CFI having the amino acid sequence set forth in SEQ ID NO: 5.

In some embodiments, the CFI variant comprises one or more modifications at one or more N-linked glycosylation sites of the CFI. In some embodiments, the CFI variant comprises a modification at any one or more positions corresponding to positions N52, N85, N159, N446, N476, and N518 in a CFI having the amino acid sequence set forth in SEQ ID NO: 5. In some embodiments, the CFI variant comprises one or more of the substitutions selected from N52Q, N85Q, N159Q, N446Q, N476Q, and N518Q, wherein the positions correspond to positions in a CFI having the amino acid sequence set forth in SEQ ID NO: 5. In some embodiments, the CFI variant comprises one or more of combination of substitutions selected from N52Q; N85Q; N159Q, N446Q; N476Q; N518Q, wherein the positions correspond to positions in a CFI having the amino acid sequence set forth in SEQ ID NO: 5.

In some embodiments, the CFI variant comprises one or more modifications in the SPD domain of the CFI. In some embodiments, the CFI variant comprises a modification at any one or more positions corresponding to positions K14, K312, R314, I322, V323, K326, R327, A328, K340, D341, G344, I345, T346, A361, L364, Y372, W381, P384, V390, N402, N404, G406, Y408, Q409, E416, K418, N422, D425, E457, K458, R456, E461, R462, F464, S465, Q467, W468, G469, T495, Y496, D497, S499, I500, A502, K504, D506, S507, E530, N531, E530, N531, G533, K534, P535, E536, and F537 in a CFI having the amino acid sequence set forth in SEQ ID NO: 5.

In some embodiments, the CFI variant comprises a substitution of an autolysis loop of the CFI for an autolysis loop of trypsin, wherein the autolysis loop occurs between positions corresponding to position 456 and position 465 in a CFI having the amino acid sequence set forth in SEQ ID NO: 5.

In some embodiments, the CFI variant comprises one or more of the substitutions selected from K14A, K312A, R314A, I322T, I322Y, I322V, V323I, V323G, V323A, K326A, R327A, R327P, R327N, A328C, K340G, D341A, G344R, G344K, G344Y, I345G, T346R, T346K, T346H, A361G, L364G, Y372G, W381K, W381G, P384A, P384G, V390G, N402E, N404G, G406D, G406E, G406F, G406H, G406I, G406K, G406L, G406M, G406N, G406P, G406Q, G406S, G406T, G406V, G406W, G406Y, Y408L, Y408F, Y408G, Y408P, Y408D, Y408A, Y408N, Y408T, Y408K, Y408R, Y408H, Y408I, Y408E, Y408M, Y408Q, Y408S, Y408W, Y408V, Q409G, E416A, K418G, N422K, D425A, D425K, D425R, D425G, R456A, R456N, E457G, E457A, E457D, E457F, E457H, E457I, E457K, E457L, E457M, E457N, E457P, E457Q, E457R, E457S, E457T, E457W, E457Y, E457V, K458A, E461Q, E461K, E461R, E461H, E461G, E461A, E461D, E461F, E461I, E461L E461M, E461N, E461P, E461S, E461T, E461W, E461Y, E461V, R462K, R462A, R462D, F464Y, S465G, Q467K, Q467R, W468C, G469L, T495F, Y496L, D497E, S499G, I500K, A502S, K504Q, K504E, K504R, K504A, K504G, K504L, K504P, K504H, K504D, K504F, K504I, K504M, K504N, K504S, K504T, K504V, K504W, K504Y, D506A, D506V, D506E, D506G, S507A, E530D, E530G, E530F, E530Y, N531G, N531A, E530D, E530G, E530F, E530Y, E530R, E530K, N531D, N531E, N531F, N531H, N531I, N531K, N531L, N531M, N531P, N531Q, N531R, N531S, N531T, N531V, N531W, N531Y, G533A, K534Q, P535A, P535K, E536N, E536A, F537K and F537R, wherein the positions correspond to positions in a CFI having the amino acid sequence set forth in SEQ ID NO: 5.

In some embodiments, the CFI variant comprises one or more of the combination substitutions selected from K326A; R327A, N531G; P535A, E457G; E461Q; R462K; F464Y, Y408L; N531G; E457G, Y408L; N531G; E457G; E461Q, Y408L; N531G; E457G; E461Q-R462K; F464Y, Y408L; N531G; P535A, K14A; D425R, E530D; N531G; G533A; K534Q; P535K; E536N, A502S; K504Q; F537K, T495F; Y496L; D497E; S499G; I500K, G533A; K534Q; P535K; E536N; F537K, T495F; Y496L; D497E; S499G; I500K; G533A; K534Q; P535K; E536N; F537K, Q467K; F537K, E530G; N531G, E530D; F537K, E457G; E461Q, E457G; E461G, Y408L; N531G; E457G; E461Q, N531G; E457G; E461Q, I322V; V323I, I322V; V323I; R327P, A328C; W468C, A328C; W468C; K326Y; R327N, Y408L; N531G; E461Q, Y408L; N531G; E457G; E461Q; R462K, Y408L; N531G; E457G; E461Q; F464Y, Y408L; N531G; E457G; R462K; F464Y, Y408L; N531G; E461Q; R462K; F464Y, Y408L; E457G; E461Q; R462K; F464Y, E457G; N531G; E461Q; R462K; F464Y, Y408L; E457G; E461Q; R462K, N531G; E457G; E461Q; F464Y, E416A; D425R, Y408L; N531G; E457G; E461Q; R462K; F464Y; S507A, E457G; E461G, K312A; R314A, G469L; R456N; E457T; K458A, G469L; R456N; K458A, G469L; R456N; K458A; E461G, G469L; R456N; K458A; E461G; F537K, G406D; Y408L, G406D; N531G, G406D; P535A, G406D; Y408L; N531G, G406D; Y408L; P535A, G406D; N531G; P535A, G406D; Y408L; N531G; P535A, K340G; I345G, L364G; Y372G, W381G; V390G, W381G; P384A; V390G, W381G; P384G; V390G, N404G; Q409G, K418G; D425G, T346R; K504E; E530R, T346K; K504D; E530K, G344R; Y408L; N531G, G344K; Y408L; N531G, T346R; Y408L; N531G, T346K; Y408L; N531G, K504D; Y408L; N531G, K504E; Y408L; N531G, Y408L; E530R; N531G, Y408L; E530K; N531G, T346R; Y408L; K504E; E530R; N531G, T346K; Y408L; K504D; E530K; N531G, Y408L; S507A; N531G, Y408L; N531G; E457G; E461Q; R462K; F464Y; S507A, E457G; S507A, and N531G; P535A; S507A, wherein the positions correspond to positions in a CFI having the amino acid sequence set forth in SEQ ID NO: 5.

In some embodiments, the CFI variant comprises one or more modifications at an active site of the CFI. In some embodiments, the CFI variant comprises a modification at a position corresponding to position S507 in a CFI having the amino acid sequence set forth in SEQ ID NO: 5. In some embodiments, the CFI variant comprises a substitution S507A, wherein the position corresponds to position S507 in a CFI having the amino acid sequence set forth in SEQ ID NO: 5.

In some embodiments, the CFI variant comprises an A chain and a B chain, wherein the CFI variant comprises a structural arrangement from N-terminus to C-terminus as (A chain)-(optional linker)-(B chain). In some embodiments, the CFI variant comprises an A chain and a B chain, wherein the CFI variant comprises a structural arrangement from N-terminus to C-terminus as (B chain)-(optional linker)-(A chain). In some embodiments, the CFI variant comprises modifications at one or more of C309 and C435, wherein the positions correspond to positions in a CFI having the amino acid sequence set forth in SEQ ID NO: 5.

In some embodiments, the CFI variant comprises substitutions C309S; C435S, wherein the positions correspond to positions in a CFI having the amino acid sequence set forth in SEQ ID NO: 5. In some embodiments, the B chain and the A chain are further linked by a disulfide bond.

In some embodiments, the CFI variant is more easily activated as compared to the wild type CFI. In some embodiments, the CFI variant comprises a modification at any one or more positions corresponding to positions I317, R318, R319, K320, and R321 in a CFI having the amino acid sequence set forth in SEQ ID NO: 5. In some embodiments, the CFI variant comprises one or more of the substitutions selected from I317D, R318D, R319D, K320D, and R321K, wherein the positions correspond to positions in a CFI having the amino acid sequence set forth in SEQ ID NO: 5.

In some embodiments, the CFI variant comprises substitutions I317D, R318D, R319D, K320D, and R321K, wherein the positions correspond to positions in a CFI having the amino acid sequence set forth in SEQ ID NO: 5.

In some embodiments, the CFI variant comprises one or more of the combination of substitutions selected from Y408; N531G, E38A; D425R, Y20F; D425R, S250A; D425R, Y408F; N531G, Y408L; N531G; E457G; E461Q; R462K; F464Y, K14A; Y20F, K14A; E38A, K14A; S250A, K14A; D425A, Y20F; E38A, Y20F; S250A, Y20F; D425A, E38A; S250A, E38A; D425A, S250A; D425A, K14A; N531G; P535A, Y20F; N531G; P535A, E38A; N531G; P535A, S250A; N531G; P535A, D425A; N531G; P535A, Y20F; Y408L; N531G; E457G; E461Q; R462K; F464Y, E38A; Y408L; N531G; E457G; E461Q; R462K; F464Y, S250A; Y408L; N531G; E457G; E461Q; R462K; F464Y, D425R; Y408L; N531G; E457G; E461Q; R462K; F464Y, Y20F; E38A; S250A; D425A, Y20F; E38A; S250A; D425A; Y408L; N531G; E457G; E461Q; R462K; F464Y, Y20F; E38A; S250A; D425A; Y408L; N531G; E457G; E461Q, I317D; R318D; R319D; K320D; R321K; E457G; E461Q-R462K; F464Y, I317D; R318D; R319D; K320D; R321K; E457G; E461Q-R462K; F464Y, I317D; R318D; R319D; K320D; R321K; Y408L; N531G; E457G; E461Q; R462K; F464Y, K504D; Y408L; N531G, K504E; Y408L; N531G, E457G; N531G; D425K, Y408F; N531G, Y408L; E457G; N531G; D425K, Y408L; E457G; P535G; D425K, Y408L; E457G; N531G; K534Q, Y408L; N531G, R462K; F464Y, and Y408L; P535G; D425K, wherein the positions correspond to positions in a CFI having the amino acid sequence set forth in SEQ ID NO: 5.

In some embodiments, the CFI variants comprise or consist of a modification at any one or more positions corresponding to positions K14, K312, R314, I322, V323, K326, R327, A328, T377, K340, D341, G344, I345, T346, A361, L364, Y372, W381, P384, V390, N402, N404, G406, Y408, Q409, E416, K418, N422, D425, E457, K458, R456, E461, R462, F464, S465, Q467, W468, G469, T495, Y496, D497, S499, I500, A502, K504, D506, S507, E530, N531, E530, N531, G533, K534, P535, E536, and F537 in a CFI having the amino acid sequence set forth in SEQ ID NO: 5.

In some embodiments, the CFI variants comprise or consist of any one or more of the substitutions selected from K14A, K312A, R314A, I322T, I322Y, I322V, V323I, V323G, V323A, K326A, R327A, R327P, R327N, A328C, T377G, K340G, D341A, G344R, G344K, G344Y, I345G, T346R, T346K, T346H, A361G, L364G, Y372G, W381K, W381G, P384A, P384G, V390G, N402E, N404G, G406D, G406E, G406F, G406H, G406I, G406K, G406L, G406M, G406N, G406P, G406Q, G406S, G406T, G406V, G406W, G406Y, Y408L, Y408F, Y408G, Y408P, Y408D, Y408A, Y408N, Y408T, Y408K, Y408R, Y408H, Y408I, Y408E, Y408M, Y408Q, Y408S, Y408W, Y408Y, Y408V, Q409G, E416A, K418G, N422K, D425A, D425K, D425R, D425G, R456A, R456N, E457G, E457A, E457D, E457F, E457H, E457I, E457K, E457L, E457M, E457N, E457P, E457Q, E457R, E457S, E457T, E457W, E457Y, E457V, K458A, E461Q, E461K, E461R, E461H, E461G, E461A, E461D, E461F, E461I, E461L E461M, E461N, E461P, E461S, E461T, E461W, E461Y, E461V, R462K, R462A, R462D, F464Y, S465G, Q467K, Q467R, W468C, G469L, T495F, Y496L, D497E, S499G, I500K, A502S, K504Q, K504E, K504R, K504A, K504G, K504L, K504P, K504H, K504D, K504F, K504I, K504M, K504N, K504S, K504T, K504V, K504W, K504Y, D506A, D506V, D506E, D506G, S507A, E530D, E530G, E530F, E530Y, N531G, N531A, E530D, E530G, E530F, E530Y, E530R, E530K, N531D, N531E, N531F, N531H, N531I, N531K, N531L, N531M, N531P, N531Q, N531R, N531S, N531T, N531V, N531W, N531Y, G533A, K534Q, P535A, P535K, E536N, E536A, F537K and F537R, wherein the positions correspond to positions in a CFI having the amino acid sequence set forth in SEQ ID NO: 5.

In some embodiments, the CFI variants comprise or consist of any one or more of the combination substitutions selected from N422K+E457G+N531G, T377G+E457G+N531G, K326A+327A R327A, E457G+N531G, N531G+P535A, E457G+E461Q+R462K+F464Y, Y408L+N531G+E457G, Y408L+N531G+E457G+E461Q, Y408L+N531G+E457G+E461Q-R462K+F464Y, Y408L+N531G+P535A, K14A+D425R, E530D+N531G+G533A+K534Q+P535K+E536N, A502S+K504Q+F537K, T495F+Y496L+D497E+S499G+I500K, G533A+K534Q+P535K+E536N+F537K, T495F+Y496L+D497E+S499G+I500K+G533A+K534Q+P535K+E536N+F537K, Q467K+F537K, E530G+N531G, E530D+F537K, E457G+E461Q, E457G+E461G, Y408L+N531G+E457G+E461Q, N531G+E457G+E461Q, I322V+V323I, I322V+V323I+R327P, A328C+W468C, A328C+W468C+K326Y+R327N, Y408L+N531G+E461Q, Y408L+N531G+E457G+E461Q+R462K, Y408L+N531G+E457G+E461Q+F464Y, Y408L+N531G+E457G+R462K+F464Y, Y408L+N531G+E461Q+R462K+F464Y, Y408L+E457G+E461Q+R462K+F464Y, E457G+N531G+E461Q+R462K+F464Y, Y408L+E457G+E461Q+R462K, N531G+E457G+E461Q+F464Y, E416A+D425R, Y408L+N531G+E457G+E461Q+R462K+F464Y+S507A, E457G+E461G, K312A+R314A, G469L+R456N+E457T+K458A, G469L+R456N+K458A, G469L+R456N+K458A+E461G, G469L+R456N+K458A+E461G+F537K, G406D+Y408L, G406D+N531G, G406D+P535A, G406D+Y408L+N531G, G406D+Y408L+P535A, G406D+N531G+P535A, G406D+Y408L+N531G+P535A, K340G+I345G, L364G+Y372G, W381G+V390G, W381G+P384A+V390G, W381G+P384G+V390G, N404G+Q409G, K418G+D425G, T346R+K504E+E530R, T346K+K504D+E530K, G344R+Y408L+N531G, G344K+Y408L+N531G, T346R+Y408L+N531G, T346K+Y408L+N531G, K504D+Y408L+N531G, K504E+Y408L+N531G, Y408L+E530R+N531G, Y408L+E530K+N531G, T346R+Y408L+K504E+E530R+N531G, T346K+Y408L+K504D+E530K+N531G, Y408L+S507A+N531G, Y408L+N531G+E457G+E461Q+R462K+F464Y+S507A, E457G+S507A, and N531G+P535A+S507A, wherein the positions correspond to positions in a CFI having the amino acid sequence set forth in SEQ ID NO: 5. For example, in some embodiments, the CFI variant comprises or consists of substitutions selected from N422K+E457G+N531G. For example, in some embodiments, the CFI variant comprises or consists of substitutions selected from T377G+E457G+N531G.

In some embodiments, the fusion construct comprises CFI and albumin, and the albumin is human serum albumin (HSA), and the CFI is a wild type CFI, and such fusion constructs are referred to herein as “CFI-HSA.” An exemplary CFI-HSA of the disclosure has the amino acid sequence of SEQ ID NO: 21, or a sequence comprising at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the amino acid sequence of SEQ ID NO. 21.

In some embodiments, the CFI of the fusion construct comprises the amino acid sequence of SEQ ID NO. 5, or a variant thereof. In some embodiments, the variant comprises at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the amino acid sequence of SEQ ID NO. 5.

In some embodiments, the serum albumin of the fusion construct comprises the amino acid sequence of SEQ ID NO. 7 or a variant thereof. In some embodiments, the variant comprises at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the amino acid sequence of SEQ ID NO. 7.

In some embodiments, the binding partner of CFI in the fusion construct comprises at least one domain, or part of a domain of Complement Receptor 1 (CR1). In some embodiments, the at least one CR1 domain comprises CCP domains 15-17 of CR1. In some embodiments, the at least one CR1 domain comprises CCP domains 1-3 of CR1. In some embodiments, the fusion constructs of the disclosure comprising at least one CR1 domain also include fusion with albumin. In some embodiments, the fusion constructs of the disclosure comprising at least one CR1 domain also include fusion with albumin, and/or at least one domain of Factor H. In some embodiments, the at least one CR1 domain comprises CR1 CCP domain 15. In some embodiments, the at least one CR1 domain comprises CR1 CCP domain 16. In some embodiments, the at least one CR1 domain comprises CR1 CCP domain 17. In some embodiments, the at least one CR1 domain comprises CR1 CCP domains 15-16. In some embodiments, the at least one CR1 domain comprises CR1 CCP domains 16-17. In some embodiments, an exemplary fusion construct comprises a CFI having the modification N531G fused with CCP domains 15-17 of CR1. In some embodiments, the exemplary fusion construct comprises a CFI fused with CCP domains of CR1, and is further fused with albumin.

In some embodiments, the binding partner of CFI in the fusion construct comprises at least one domain, or part of a domain of Factor H. In some embodiments, the at least one Factor H domain comprises any one or more of complement control protein (CCP) domains 1-20 of Factor H. In some embodiments, the amino acid sequence of the at least one Factor H domain is, or is derived from, the sequence set forth in SEQ ID NO: 22 (Table 1). In some embodiments, the at least one Factor H domain comprises each of the CCP domains 1-20 of Factor H. In some embodiments, the at least one Factor H domain comprises CCP1, CCP 2, CCP3, and CCP4. In some embodiments, the at least one Factor H domain comprises CCP2, CCP3, and CCP4. In some embodiments, the at least one Factor H domain comprises CCP2 and CCP3. In some embodiments, the amino acid sequence of the at least one domain of Factor H is, or is derived from, the sequence set forth in SEQ ID NO: 23 (Table 1). In some embodiments, the at least one Factor H domain comprises CCP domains 1-4 and 19-20 of Factor H. In some embodiments, the exemplary fusion construct comprises a CFI fused with CCP domains of FH, and is further fused with albumin.

Exemplary CFI-HSA fusion constructs of the disclosure include, as represented N-terminal to C-terminal:
(a) N terminal HSA—GGSSGG linker (SEQ ID NO: 6)—CFI SEQ ID NO: 5 comprising substitutions N422K, E457G and N531G.
(b) N-terminal HSA—GGSSGG linker (SEQ ID NO: 6)—CFI SEQ ID NO: 5 comprising substitutions T377G, E457G and N531G.

Exemplary CR1-containing fusion constructs of the disclosure include HSA, CFI, and human CR1 domains. An example of such a fusion construct is:
(a) N-terminal HSA—GGSSGG linker (SEQ ID NO: 6)—CFI SEQ ID NO: 5 comprising substitution N422K, E457G, and N531G—GGSSGGSSGGSSGGSSGGSSGG linker (SEQ ID NO: 24)—human CR1 domains CCP15, CCP16, and CCP17.

The components of the fusion constructs (the CFI and the binding partner) of the disclosure may be held together by optional linkers. They may be of any suitable length of at least one amino acid. A linker may be a flexible linker, and may be a peptide of about 1 to about 20 amino acid residues in length, wherein the amino acid residues may comprise glycine residues. The linker may also optionally comprise serine residues. Exemplary flexible linkers can include, but are not limited to, glycine polymers, glycine-serine polymers, glycine-alanine polymers, alanine-serine polymers, or any other suitable flexible linkers known in the art. An exemplary linker is (GGSS)ⁿGG (SEQ ID NO: 25), wherein n is any number from about 1 to about 25. In some embodiments, the linkers are protease-sensitive cleavable linkers. Exemplary linkers linking the fusion constructs can be 1-50, 5-50, 10-50, 15-50, 20-22, 22-24, 20-50, 25-50, 1-20, 2-20, 3-20, 4-20, 5-20, 6-20, 7-20, 8-20, 9-20, 10-20, 3-15, 3-10, 3-9, 3-8, 3-7, 3-6, 3-5, 4-15, 4-10, 4-9, 4-8, 4-7, 4-6, 4-5, 5-15, 5-10, 5-9, 5-8, 5-7, 5-6, 6-15, 6-10, 6-9, 6-8, or 6-7 amino acids in length.

In some embodiments, a CFI-HSA may have an extended half-life with respect to a CFI not part of a fusion construct. An exemplary CFI-HSA construct can be generated by linking an albumin with wild type CFI by a flexible linker. In some embodiments, the CFI-HSA comprises the amino acid sequence set forth in SEQ ID NO: 21, or comprises a sequence having at least 80%, 85%, 90%, 95%, 96%, 97%, 98%, or 99% amino acid sequence identity thereto.

In some embodiments, the fusion construct comprises a structural arrangement from N-terminus to C-terminus as (Albumin)-(optional linker)-(WT CFI).

In some embodiments, the fusion construct comprises a structural arrangement from N-terminus to C-terminus as (WT CFI)-(optional linker)-(Albumin).

In some embodiments, the fusion construct comprises the amino acid sequence of SEQ ID NO: 7 and SEQ ID NO: 5, or a variant thereof that has at least 70%, at least 75%, at least 80%, at least 85%, at least 90%, at least 95%, or at least 99% identity with SEQ ID NO.5, wherein the fusion construct comprises a structural arrangement from N-terminus to C-terminus (SEQ ID NO: 7)-(optional linker)-(SEQ ID NO: 5). In some embodiments, the fusion construct comprises the amino acid sequence of SEQ ID NO: 5 and SEQ ID NO: 7 wherein the fusion construct comprises a structural arrangement from N-terminus to C-terminus (SEQ ID NO: 7)-(linker)-(SEQ ID NO: 5). In some embodiments, the fusion construct comprises the amino acid sequence of SEQ ID NO: 5, SEQ ID NO: 6, and SEQ ID NO: 7, wherein the fusion construct comprises a structural arrangement from N-terminus to C-terminus (SEQ ID NO: 7)-(SEQ ID NO: 6)-(SEQ ID NO: 5). In some embodiments, the fusion construct comprises the amino acid sequence of SEQ ID NO: 5 and SEQ ID NO: 7 wherein the fusion construct comprises a structural arrangement from N-terminus to C-terminus (SEQ ID NO: 5)-(optional linker)-(SEQ ID NO: 7). In some embodiments, the fusion construct comprises the amino acid sequence of SEQ ID NO: 5 and SEQ ID NO: 7 wherein the fusion construct comprises a structural arrangement from N-terminus to C-terminus (SEQ ID NO: 5)-(linker)-(SEQ ID NO: 7). In some embodiments, the fusion construct comprises the amino acid sequence of SEQ ID NO: 5, SEQ ID NO: 6, and SEQ ID NO: 7, wherein the fusion construct comprises a structural arrangement from N-terminus to C-terminus (SEQ ID NO: 5)-(SEQ ID NO: 6)-(SEQ ID NO: 7).

In some embodiments, the fusion construct comprises an amino acid sequence set forth in SEQ ID NO: 21, or an amino acid sequence comprising at least 70%, at least 80%, at least 90%, at least 95%, or at least 99% identity thereto. In some embodiments, the fusion construct consists of an amino acid sequence set forth in SEQ ID NO: 21. In some embodiments, the fusion construct comprises the amino acid sequence of SEQ ID NO: 5 and SEQ ID NO: 7. In some embodiments it is noted that albumin fusion (e.g. N-terminal albumin fusion) to a wild type CFI provides solubility and facilitates activation of CFI-HSA. When activation of CFI to the mature two-chain protein with furin is carried out post translationally and activation is compared between CFI-HSA and a wild type CFI without an albumin (WT-CFI), it is observed that furin activates the CFI-HSA significantly better, and almost completely. It is observed that the CFI-HSA protein remains as a monomer with no evidence of aggregates. There is a significant and unexpected benefit of the amino terminal HSA fusion for maintaining solubility, monodispersity and efficient furin activation of a CFI-HSA construct. There is a significant improvement of bioavailability through, for example, improved half-life.

Accordingly provided herein are methods of increasing the activation of a CFI, comprising fusing a HSA to a wild type CFI, wherein the fusion is a N-terminal fusion prior to activation with furin; and activating with furin. In some embodiments the activation with furin is carried out in a cell during recombinant production of CFI fusion construct of the disclosure. In some embodiments the activation with furin is carried out in vitro.

Turning to Table 1, SEQ ID NO: 1 is the amino acid sequence of wild type plasma-derived human CFI, referred to as “CFI-PD”, and has a leader sequence. Wild type CFI used for fusion with a second component may comprise the amino acid sequence of SEQ ID NO: 5, which does not include the leader sequence present in SEQ ID NO: 1. A mouse Ig kappa chain V-Ill region MOPC 63 leader sequence (SEQ ID NO: 2) may instead be used for the recombinant production of any of the CFI fusion constructs provided herein. In some embodiments, provided herein are CFI fusion constructs comprising at least one CFI domain, wherein the at least one CFI domain comprises the amino acid sequence set forth in SEQ ID NO: 5.

TABLE 1
Components of Exemplary CFI Fusion Constructs

| Description | Sequence |
|---|---|
| Wild type | MKLLHVFLLFLCFHLRFCKVTYTSQEDLVEKKCLAKKYTHLSCD |
| plasma-derived | KVFCQPWQRCIEGTCVCKLPYQCPKNGTAVCATNRRSFPTYCQQ |
| human CFI | KSLECLHPGTKFLNNGTCTAEGKFSVSLKHGNTDSEGIVEVKLV |
| (CFI-PD) | DQDKTMFICKSSWSMREANVACLDLGFQQGADTQRRFKLSDLSI |
| | NSTECLHVHCRGLETSLAECTFTKRRTMGYQDFADVVCYTQKAD |
| | SPMDDFFQCVNGKYISQMKACDGINDCGDQSDELCCKACQGKGF |
| | HCKSGVCIPSQYQCNGEVDCITGEDEVGCAGFASVTQEETEILT |
| | ADMDAERRRIKSLLPKLSCGVKNRMHIRRKRIVGGKRAQLGDLP |
| | WQVAIKDASGITCGGIYIGGCWILTAAHCLRASKTHRYQIWTTV |
| | VDWIHPDLKRIVIEYVDRIIFHENYNAGTYQNDIALIEMKKDGN |
| | KKDCELPRSIPACVPWSPYLFQPNDTCIVSGWGREKDNERVFSL |
| | QWGEVKLISNCSKFYGNRFYEKEMECAGTYDGSIDACKGDSGGP |
| | LVCMDANNVTYVWGVVSWGENCGKPEFPGVYTKVANYFDWISYH |
| | VGRPFISQYNV (SEQ ID NO: 1) |
| Leader | METDTLLLWVLLLWVPGSTG (SEQ ID NO: 2) |
| sequence | |
| (mouse leader | |
| for CFI-HSA) | |
| Human CFI | MKLLHVFLLFLCFHLRFC (SEQ ID NO: 3) |
| leader | |
| sequence | |
| Wild type CFI | KVTYTSQEDLVEKKCLAKKYTHLSCDKVFCQPWQRCIEGTCVCK |
| of SEQ ID NO | LPYQCPKNGTAVCATNRRSFPTYCQQKSLECLHPGTKFLNNGTC |
| 1 without | TAEGKFSVSLKHGNTDSEGIVEVKLVDQDKTMFICKSSWSMREA |
| signal | NVACLDLGFQQGADTQRRFKLSDLSINSTECLHVHCRGLETSLA |
| sequence | ECTFTKRRTMGYQDFADVVCYTQKADSPMDDFFQCVNGKYISQM |
| | KACDGINDCGDQSDELCCKACQGKGFHCKSGVCIPSQYQCNGEV |
| | DCITGEDEVGCAGFASVTQEETEILTADMDAERRRIKSLLPKLS |
| | CGVKNRMHIRRKRIVGGKRAQLGDLPWQVAIKDASGITCGGIYI |
| | GGCWILTAAHCLRASKTHRYQIWTTVVDWIHPDLKRIVIEYVDR |
| | IIFHENYNAGTYQNDIALIEMKKDGNKKDCELPRSIPACVPWSP |
| | YLFQPNDTCIVSGWGREKDNERVFSLQWGEVKLISNCSKFYGNR |
| | FYEKEMECAGTYDGSIDACKGDSGGPLVCMDANNVTYVWGVVSW |
| | GENCGKPEFPGVYTKVANYFDWISYHVGRPFISQYNV |
| | (SEQ ID NO: 5) |
| Linker | GGSSGG (SEQ ID NO: 6) |
| Human serum | DAHKSEVAHRFKDLGEENFKALVLIAFAQYLQQCPFEDHVKLVN |
| albumin (HSA) | EVTEFAKTCVADESAENCDKSLHTLFGDKLCTVATLRETYGEMA |
| | DCCAKQEPERNECFLQHKDDNPNLPRLVRPEVDVMCTAFHDNEE |
| | TFLKKYLYEIARRHPYFYAPELLFFAKRYKAAFTECCQAADKAA |
| | CLLPKLDELRDEGKASSAKQRLKCASLQKFGERAFKAWAVARLS |
| | QRFPKAEFAEVSKLVTDLTKVHTECCHGDLLECADDRADLAKYI |
| | CENQDSISSKLKECCEKPLLEKSHCIAEVENDEMPADLPSLAAD |
| | FVESKDVCKNYAEAKDVFLGMFLYEYARRHPDYSVVLLLRLAKT |
| | YETTLEKCCAAADPHECYAKVFDEFKPLVEEPQNLIKQNCELFE |
| | QLGEYKFQNALLVRYTKKVPQVSTPTLVEVSRNLGKVGSKCCKH |
| | PEAKRMPCAEDYLSVVLNQLCVLHEKTPVSDRVTKCCTESLVNR |
| | RPCFSALEVDETYVPKEFNAETFTFHADICTLSEKERQIKKQTA |
| | LVELVKHKPKATKEQLKAVMDDFAAFVEKCCKADDKETCFAEEG |
| | KKLVAASQAALGL (SEQ ID NO: 7) |
| HSA linked | DAHKSEVAHRFKDLGEENFKALVLIAFAQYLQQCPFEDHVKLVN |
| with CFI (CFI- | EVTEFAKTCVADESAENCDKSLHTLFGDKLCTVATLRETYGEMA |
| HSA) | DCCAKQEPERNECFLQHKDDNPNLPRLVRPEVDVMCTAFHDNEE |
| | TFLKKYLYEIARRHPYFYAPELLFFAKRYKAAFTECCQAADKAA |
| | CLLPKLDELRDEGKASSAKQRLKCASLQKFGERAFKAWAVARLS |
| | QRFPKAEFAEVSKLVTDLTKVHTECCHGDLLECADDRADLAKYI |
| | CENQDSISSKLKECCEKPLLEKSHCIAEVENDEMPADLPSLAAD |
| | FVESKDVCKNYAEAKDVFLGMFLYEYARRHPDYSVVLLLRLAKT |
| | YETTLEKCCAAADPHECYAKVFDEFKPLVEEPQNLIKQNCELFE |
| | QLGEYKFQNALLVRYTKKVPQVSTPTLVEVSRNLGKVGSKCCKH |
| | PEAKRMPCAEDYLSVVLNQLCVLHEKTPVSDRVTKCCTESLVNR |
| | RPCFSALEVDETYVPKEFNAETFTFHADICTLSEKERQIKKQTA |
| | LVELVKHKPKATKEQLKAVMDDFAAFVEKCCKADDKETCFAEEG |
| | KKLVAASQAALGLGGSSGGKVTYTSQEDLVEKKCLAKKYTHLSC |
| | DKVFCQPWQRCIEGTCVCKLPYQCPKNGTAVCATNRRSFPTYCQ |
| | QKSLECLHPGTKFLNNGTCTAEGKFSVSLKHGNTDSEGIVEVKL |
| | VDQDKTMFICKSSWSMREANVACLDLGFQQGADTQRRFKLSDLS |
| | INSTECLHVHCRGLETSLAECTFTKRRTMGYQDFADVVCYTQKA |
| | DSPMDDFFQCVNGKYISQMKACDGINDCGDQSDELCCKACQGKG |
| | FHCKSGVCIPSQYQCNGEVDCITGEDEVGCAGFASVTQEETEIL |
| | TADMDAERRRIKSLLPKLSCGVKNRMHIRRKRIVGGKRAQLGDL |
| | PWQVAIKDASGITCGGIYIGGCWILTAAHCLRASKTHRYQIWTT |
| | VVDWIHPDLKRIVIEYVDRIIFHENYNAGTYQNDIALIEMKKDG |
| | NKKDCELPRSIPACVPWSPYLFQPNDTCIVSGWGREKDNERVFS |
| | LQWGEVKLISNCSKFYGNRFYEKEMECAGTYDGSIDACKGDSGG |
| | PLVCMDANNVTYVWGVVSWGENCGKPEFPGVYTKVANYFDWISY |
| | HVGRPFISQYNV (SEQ ID NO: 21) |
| Human Factor H | MRLLAKIICLMLWAICVAEDCNELPPRRNTEILTGSWSDQTYPE |
| (FH) | GTQAIYKCRPGYRSLGNVIMVCRKGEWVALNPLRKCQKRPCGHP |
| | GDTPFGTFTLTGGNVFEYGVKAVYTCNEGYQLLGEINYRECDTD |
| | GWTNDIPICEVVKCLPVTAPENGKIVSSAMEPDREYHFGQAVRF |
| | VCNSGYKIEGDEEMHCSDDGFWSKEKPKCVEISCKSPDVINGSP |
| | ISQKIIYKENERFQYKCNMGYEYSERGDAVCTESGWRPLPSCEE |
| | KSCDNPYIPNGDYSPLRIKHRTGDEITYQCRNGFYPATRGNTAK |
| | CTSTGWIPAPRCTLKPCDYPDIKHGGLYHENMRRPYFPVAVGKY |
| | YSYYCDEHFETPSGSYWDHIHCTQDGWSPAVPCLRKCYFPYLEN |
| | GYNQNYGRKFVQGKSIDVACHPGYALPKAQTTVTCMENGWSPTP |
| | RCIRVKTCSKSSIDIENGFISESQYTYALKEKAKYQCKLGYVTA |
| | DGETSGSITCGKDGWSAQPTCIKSCDIPVFMNARTKNDFTWFKL |
| | NDTLDYECHDGYESNTGSTTGSIVCGYNGWSDLPICYERECELP |
| | KIDVHLVPDRKKDQYKVGEVLKFSCKPGFTIVGPNSVQCYHFGL |
| | SPDLPICKEQVQSCGPPPELLNGNVKEKTKEEYGHSEVVEYYCN |
| | PRFLMKGPNKIQCVDGEWTTLPVCIVEESTCGDIPELEHGWAQL |
| | SSPPYYYGDSVEFNCSESFTMIGHRSITCIHGVWTQLPQCVAID |
| | KLKKCKSSNLIILEEHLKNKKEFDHNSNIRYRCRGKEGWIHTVC |
| | INGRWDPEVNCSMAQIQLCPPPPQIPNSHNMTTTLNYRDGEKVS |
| | VLCQENYLIQEGEEITCKDGRWQSIPLCVEKIPCSQPPQIEHGT |
| | INSSRSSQESYAHGTKLSYTCEGGFRISEENETTCYMGKWSSPP |
| | QCEGLPCKSPPEISHGVVAHMSDSYQYGEEVTYKCFEGFGIDGP |
| | AIAKCLGEKWSHPPSCIKTDCLSLPSFENAIPMGEKKDVYKAGE |
| | QVTYTCATYYKMDGASNVTCINSRWTGRPTCRDTSCVNPPTVQN |
| | AYIVSRQMSKYPSGERVRYQCRSPYEMFGDEEVMCLNGNWTEPP |
| | QCKDSTGKCGPPPPIDNGDITSFPLSVYAPASSVEYQCONLYQL |
| | EGNKRITCRNGQWSEPPKCLHPCVISREIMENYNIALRWTAKQK |
| | LYSRTGESVEFVCKRGYRLSSRSHTLRTTCWDGKLEYPTCAKR |
| | (SEQ ID NO: 22) |
| Human mini | MRLLAKIICLMLWAICVAEDCNELPPRRNTEILTGSWSDQTYPE |
| Factor H (mini | GTQAIYKCRPGYRSLGNIIMVCRKGEWVALNPLRKCQKRPCGHP |
| FH) | GDTPFGTFTLTGGNVFEYGVKAVYTCNEGYQLLGEINYRECDTD |
| | GWTNDIPICEVVKCLPVTAPENGKIVSSAMEPDREYHFGQAVRF |
| | VCNSGYKIEGDEEMHCSDDGFWSKEKPKCVEISCKSPDVINGSP |
| | ISQKIIYKENERFQYKCNMGYEYSERGDAVCTESGWRPLPSCEE |
| | AGGGGGGGGGGGGGKCGPPPPIDNGDITSFPLSVYAPASSVEYQ |
| | CONLYQLEGNKRITCRNGQWSEPPKCLHPCVISREIMENYNIAL |
| | RWTAKQKLYSRTGESVEFVCKRGYRLSSRSHTLRTTCWDGKLEY |
| | PTCAKRENLYFQGHHHHHH |
| | (SEQ ID NO: 23) |

### II. Generation of CFI Fusion Constructs

Cell lines can be developed to express production of the CFI fusion constructs described herein. Cell lines for producing the fusion constructs can be produced using any host cell capable of expressing the fusion constructs described herein. Host cells can be mammalian cells, insect cells, fungal cells, plant cells, and/or bacterial cells. For expression of the CFI fusion constructs, the host cell line can be transiently or stably transfected or transduced with expression vectors encoding the fusion constructs. Vectors can be, for example, plasmids or viral vectors. In some embodiments, the host cell line is a mammalian cell line. In some embodiments, the host cell is a Chinese hamster ovary (CHO) cell.

CFI fusion constructs described herein can be recombinantly expressed in mammalian cell lines known in the art for producing biologic products, e.g. Chinese hamster ovary (CHO) cells. Mammalian cells can be transfected or transduced with an expression vector encoding the fusion constructs described herein using any method known in the art.

Provided herein are methods for production and purification of CFI fusion constructs described herein. CFI fusion constructs described herein may be purified from conditioned media by standard methods known in the art. In some embodiments CFI fusion constructs may be purified by chromatography on affinity matrices. In some embodiments the affinity matrix is CaptureSelect™ human albumin affinity matrix. In some embodiments CFI fusion constructs may be purified by chromatography on cation and/or anion exchange matrices and optionally size exclusion chromatography. CFI fusion constructs may optimally be buffer exchanged into any suitable buffer known in the art. Purity can be assessed by any method known in the art including gel electrophoresis, orthogonal HPLC methods, staining and spectrophotometric techniques.

### III. CFI Fusion Constructs—Methods of Use

### A. Uses

The CFI fusion constructs of the disclosure may be used for modulating the complement system in the eye.

As discussed herein, in some embodiments, a CFI fusion construct of the disclosure is capable of modulating the classical and lectin complement pathway in the eye.

As discussed herein, in some embodiments, a CFI fusion construct of the disclosure is capable of modulating the alternate complement pathway in the eye.

As discussed herein, in some embodiments, a CFI fusion construct of the disclosure is capable of decreasing the amplification of the complement system in the eye.

As discussed herein, in some embodiments, a CFI fusion construct of the disclosure is capable of increasing the cleavage of C3b in the eye.

As discussed herein, in some embodiments, a CFI fusion construct of the disclosure is capable of increasing the cleavage of C4b in the eye.

As discussed herein, in some embodiments, a CFI fusion construct of the disclosure is capable of increasing the generation of C4c in the eye.

As discussed herein, in some embodiments, a CFI fusion construct of the disclosure is capable of increasing the generation of iC3b in the eye.

As discussed herein, in some embodiments, a CFI fusion construct of the disclosure is capable of increasing the generation of C3dg from iC3b in the eye.

As discussed herein, in some embodiments, a CFI fusion construct of the disclosure is capable of increasing the generation of C3c from iC3b in the eye.

As discussed herein, in some embodiments, a CFI fusion construct of the disclosure is capable of reducing the level of C3b α-chain in the eye.

As discussed herein, in some embodiments, a CFI fusion construct of the disclosure is capable of increasing the hydrolysis of a peptide substrate in the eye.

As discussed herein, in some embodiments, a CFI fusion construct of the disclosure is capable of increasing the proteolysis of a macromolecular protein substrate in the eye.

As discussed herein, in some embodiments, a CFI fusion construct of the disclosure is capable of reducing in the level or function of membrane attack complex (MAC) in the eye.

As discussed herein, in some embodiments, a CFI fusion construct of the disclosure is capable reducing observed hemolysis in the eye.

As discussed herein, in some embodiments, a CFI fusion construct of the disclosure is capable of increasing the cleavage of C3b in the absence of cofactor in the eye, e.g. in a cofactor independent manner.

As discussed herein, in some embodiments, a CFI fusion construct of the disclosure is capable of increasing the cleavage of C4b in the absence of cofactor in the eye, e.g. in a cofactor independent manner.

The CFI fusion constructs of the disclosure may be used for ocular therapies in a subject. As used herein, a subject includes any mammalian subject and includes primates, rodents, domestic animals, zoo animals, and pets. In some embodiments, the mammalian subject is a human subject. In some embodiments, the mammalian subject is a non-human primate.

### B. Treatment of Ocular Conditions

The CFI fusion constructs provided herein are useful for treating an ocular condition in a subject. In some embodiments, provided herein is a method of treating an ocular condition in a subject in need thereof, the method comprising intraocularly administering to the subject a therapeutically effective amount of any of the CFI fusion constructs provided herein, or a pharmaceutical composition thereof, at a dose of about 0.1 to about 10 mg per eye and/or at a frequency of about every 1 to about 4 months.

In some embodiments, the ocular condition is characterized by a deficiency of CFI. In some embodiments, the ocular condition is characterized by dysregulation of the complement system.

In some embodiments, the ocular condition is characterized by the presence of a dysfunctional CFI gene. In some embodiments, the ocular condition is characterized by dysregulation of the complement system and low CFI levels.

In some embodiments, the ocular condition is selected from the group consisting of: diabetic macular edema (DME), diabetic retinopathy, glaucoma, keratoconjunctivitis, neuromyelitis optica spectrum disorder (NMOSD), open angle glaucoma, polypoidal choroidal vasculopathy, Stargardt Disease, uveitis, and vitreoretinopathy, and age-related macular degeneration (AMD).

In some embodiments, the ocular condition is dry AMD. In some embodiments, the ocular condition is early-stage dry AMD. In some embodiments, the ocular condition is intermediate-stage dry AMD. In some embodiments, the ocular condition is late-stage dry AMD. In some embodiments, the ocular condition is late-stage dry AMD that has progressed to wet AMD. In some embodiments, the ocular condition is late-stage dry AMD that has progressed to Geographic Atrophy (GA).

In some embodiments, the ocular condition is wet AMD. In some embodiments, the ocular condition is early-stage wet AMD. In some embodiments, the ocular condition is intermediate-stage wet AMD. In some embodiments, the ocular condition is late-stage wet AMD. In some embodiments, the ocular condition is late-stage wet AMD that has progressed to neovascular AMD.

In some embodiments, the ocular condition is geographic atrophy (GA). In some embodiments, the ocular condition is GA that has progressed from dry AMD. In some embodiments, the GA is associated with late-stage AMD. In some embodiments, the GA is not associated with late-stage AMD.

In some embodiments, the ocular condition is caused by one or more mutations to the CFI gene in the subject selected from the group consisting of: G119R, L131R, V152M, G162D, R187Y, R187T, T203I, A240G, A258T, G287R, A300T, R317W, R339Q, V412M, P553S, K441R, R339Ter, R317Q, G261D, R187Q, and R187Ter.

In some embodiments, the ocular condition is caused by one or more mutations to the CFI gene in the subject selected from the group consisting of: Phe13Val, Val20Ile, His40Arg, Cys43Phe, Asp44Asn, Pro50Ala, Cys54Arg, Cys54*, Ile55Phe, Pro64Leu, Asn70Thr, Thr72Ser, Phe82Ser, Pro83Gln, Ser90Asn, Cys106Arg, Thr107Ala, Glu109Ala, His118Arg, Gly119Arg, Gly125Arg, Val127Ala, Val129Gly, Leu131Arg, Met138Val, Met138Ile, Trp145*, Asn151Ser, Val152Met, Gly162Asp, Asp164Val, Asn177Ile, His183Arg, Val184Met, Arg187*, Arg187Gln, Gly188Ala, Phe198Leu, Arg202Ile, Thr203Ile, Tyr206Asn, Gln217His, Ser221Tyr, Asp224Asn, Cys229Arg, Val230Met, Val230Glu, Ala240Gly, Cys247Gly, Gly248Glu, Asp249Glu, Ala258Thr, Gly261Ser, Gly261Asp, Gly263Val, Lys267Asn, Gly280Asp, Gly287Arg, Glu290Asp, Ala300Thr, Glu303Lys, Glu305*, Ile306Val, Ile306Ser, Asp310Glu, Arg317Trp, Arg317Gln, Cys327Arg, Gly328Arg, Arg339*, Arg339Gln, Ile340Thr, Gly342Glu, Arg345Gln, Gly349Arg, Val355Met, Ala356Pro, Ile357Met, Gly362Ala, Tyr369Ser, Trp374Cys, Arg389His, Trp399Arg, Pro402Ser, Asp403Asn, Arg406Cys, Arg406His, Tyr411*, Val412Met, Ile416Leu, His418Leu, Gly424Asp, Asp429Glu, Ala431Thr, Ile433Val, Ile433Thr, Lys441Arg, Arg448Cys, Ala452Ser, Trp456Leu, Tyr459Ser, Gln462His, Cys467Arg, Arg474*, Arg474Gln, Asp477His, Gly487Cys, Ile492Leu, Gly500Arg, Arg502Cys, Gly512Ser, Asp519Asn, Lys522Thr, Asp524Val, Met532Val, Asn536Lys, Trp541*, Gly542Ser, Val543Ala, Trp546*, Glu548Gln, Pro553Ser, Glu554Val, Ser570Thr, Ile578Thr, and Gln580*.

In some embodiments, the ocular condition is caused by one or more of a group of mutations in the subject with GA consisting of: C2, CFB, CFH, and C3 mutations.

### C. Combination Therapies

The administration of any one of the therapeutic CFI fusion constructs provided herein may be a monotherapy, or may be in combination with any other known drugs or treatments. The other known drugs or treatments may be for conditions associated with dysregulation of the complement system, or may be associated with a CFI deficiency.

### D. Administration

The CFI fusion constructs described herein are delivered as polypeptide-based therapies to the eye.

Such treatment as contemplated herein includes administration of a CFI fusion construct of the disclosure to the eye. Accordingly, provided herein are pharmaceutical compositions comprising the CFI fusion constructs of the disclosure.

The ocular administration of the therapeutic CFI fusion constructs described herein may be intraorbital, periocular, intraocular, intravitreal, or subretinal. Administration of the fusion constructs may be performed with any suitable excipients, carriers, or other agents to provide suitable or improved tolerance, transfer, delivery, and the like. In exemplary embodiments, administration of the CFI fusion constructs described herein is an intravitreal administration.

In some embodiments, the CFI fusion constructs, can be expressed as an inactive, single chain precursor protein, and activated by furin, a serine protease. Furin is an endoprotease that cleaves CFI at its conserved RRKR sequence (also referred to as the furin recognition sequence), resulting in a heavy and light chain connected by a disulfide bond. The furin-processed, mature, two-chain protein is the activated form of the CFI protein. As generally contemplated herein, the CFI fusion constructs described herein are delivered in an activated two chain form. However, in some instances, inactive CFI fusion constructs can be delivered in an inactive single chain form. In some embodiments, what is delivered comprises both single chain inactive and two chain active forms.

### E. Dosing

In the embodiments provided herein, the CFI fusion constructs described herein are administered to one or both eyes of a subject in need thereof in a dosage of about 0.1 mg to about 10 mg per eye, and/or at a frequency of about every 4 to 16 weeks.

The optimal dose ranges and frequencies of the disclosure are expected to maintain baseline CFI levels above that of the level in a subject suffering from an ocular disease (e.g., AMD) over 10, 15, 20, 25, 30, 35, 40, 45, 50, 55, 60, 65, 70, 75, 80, 85, 90, 95, 100, 105, 110, 115, 120, 125, 130, 135, 140, 145, or 150 days post-ocular injection of a CFI fusion construct.

In some embodiments, upon intraocular administration of a fusion CFI construct, the baseline CFI level of the subject suffering from an ocular disease is maintained above that of the baseline CFI level of a reference subject suffering from the same ocular disease, who did not receive the injection. In some embodiments, such CFI levels are maintained for at least 10, 15, 20, 25, 30, 35, 40, 45, 50, 55, 60, 65, 70, 75, 80, 85, 90, 95, 100, 105, 110, 115, 120, 125, 130, 135, 140, 145, or 150 days post-injection of CFI-HSA.

In some embodiments, the CFI fusion constructs described herein are administered to the eye of a subject in need thereof in a dosage of about 0.1 mg to about 10 mg per eye, and at a frequency of about every 4 to 16 weeks.

In some embodiments, the CFI fusion constructs are administered to one eye. In some embodiments, the CFI fusion constructs are administered to both eyes. In some embodiments, the CFI fusion constructs are administered to both eyes simultaneously. In some embodiments, the CFI fusion constructs are administered to the two eyes sequentially.

Exemplary dosages of the CFI fusion constructs described herein are about 0.1 mg to about 10 mg per eye. In some embodiments, dosages of the CFI fusion constructs described herein are about 0.5 mg to about 10 mg per eye, about 1 mg to about 10 mg per eye, about 1.5 mg to about 10 mg per eye, about 2 mg to about 10 mg per eye, about 2.5 mg to about 10 mg per eye, about 3 mg to about 10 mg per eye, about 3.5 mg to about 10 mg per eye, about 4 mg to about 10 mg per eye, about 4.5 mg to about 10 mg per eye, about 5 mg to about 10 mg per eye, about 5.5 mg to about 10 mg per eye, about 6 mg to about 10 mg per eye, about 6.5 mg to about 10 mg per eye, about 7 mg to about 10 mg per eye, about 7.5 mg to about 10 mg per eye, about 8 mg to about 10 mg per eye, about 8.5 mg to about 10 mg per eye, about 9 mg to about 10 mg per eye, or about 9.5 mg to about 10 mg per eye.

In some embodiments, administration of the CFI fusion constructs described herein is an intraocular (e.g. intravitreal) administration, at a dosage of about 0.1 mg per eye, about 0.5 mg per eye, about 1 mg per eye, about 1.5 mg per eye, about 2 mg per eye, about 2.5 mg per eye, about 3 mg per eye, about 3.5 mg per eye, about 4 mg per eye, about 4.5 mg per eye, about 5 mg per eye, about 5.5 mg per eye, about 6 mg per eye, about 6.5 mg per eye, about 7 mg per eye, about 7.5 mg per eye, about 8 mg per eye, about 8.5 mg per eye, about 9 mg per eye, about 9.5 mg per eye, or about 10 mg per eye.

In some embodiments, the frequency of administration of the CFI fusion constructs is about every 4 to about every 16 weeks. In some embodiments, administration of the CFI fusion constructs is an intravitreal administration, and is about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In some embodiments, administration of the therapeutic CFI fusion constructs described herein is an intraocular (e.g. intravitreal) administration, at a dosage of about 0.1 mg per eye, about 0.5 mg per eye, about 1 mg per eye, about 1.5 mg per eye, about 2 mg per eye, about 2.5 mg per eye, about 3 mg per eye, about 3.5 mg per eye, about 4 mg per eye, about 4.5 mg per eye, about 5 mg per eye, about 5.5 mg per eye, about 6 mg per eye, about 6.5 mg per eye, about 7 mg per eye, about 7.5 mg per eye, about 8 mg per eye, about 8.5 mg per eye, about 9 mg per eye, about 9.5 mg per eye, or about 10 mg per eye, at a frequency of about every 4 weeks.

In some embodiments, administration of the CFI fusion constructs is an intraocular (e.g. intravitreal) administration, at a dosage of about 0.1 mg per eye, about 0.5 mg per eye, about 1 mg per eye, about 1.5 mg per eye, about 2 mg per eye, about 2.5 mg per eye, about 3 mg per eye, about 3.5 mg per eye, about 4 mg per eye, about 4.5 mg per eye, about 5 mg per eye, about 5.5 mg per eye, about 6 mg per eye, about 6.5 mg per eye, about 7 mg per eye, about 7.5 mg per eye, about 8 mg per eye, about 8.5 mg per eye, about 9 mg per eye, about 9.5 mg per eye, or about 10 mg per eye, at a frequency of about every 5 weeks.

In some embodiments, administration of the CFI fusion constructs is an intraocular (e.g. intravitreal) administration, at a dosage of about 0.1 mg per eye, about 0.5 mg per eye, about 1 mg per eye, about 1.5 mg per eye, about 2 mg per eye, about 2.5 mg per eye, about 3 mg per eye, about 3.5 mg per eye, about 4 mg per eye, about 4.5 mg per eye, about 5 mg per eye, about 5.5 mg per eye, about 6 mg per eye, about 6.5 mg per eye, about 7 mg per eye, about 7.5 mg per eye, about 8 mg per eye, about 8.5 mg per eye, about 9 mg per eye, about 9.5 mg per eye, or about 10 mg per eye, at a frequency of about every 6 weeks.

In some embodiments, administration of the CFI fusion constructs is an intraocular (e.g. intravitreal) administration, at a dosage of about 0.1 mg per eye, about 0.5 mg per eye, about 1 mg per eye, about 1.5 mg per eye, about 2 mg per eye, about 2.5 mg per eye, about 3 mg per eye, about 3.5 mg per eye, about 4 mg per eye, about 4.5 mg per eye, about 5 mg per eye, about 5.5 mg per eye, about 6 mg per eye, about 6.5 mg per eye, about 7 mg per eye, about 7.5 mg per eye, about 8 mg per eye, about 8.5 mg per eye, about 9 mg per eye, about 9.5 mg per eye, or about 10 mg per eye, at a frequency of about every 7 weeks.

In some embodiments, administration of the CFI fusion constructs is an intraocular (e.g. intravitreal) administration, at a dosage of about 0.1 mg per eye, about 0.5 mg per eye, about 1 mg per eye, about 1.5 mg per eye, about 2 mg per eye, about 2.5 mg per eye, about 3 mg per eye, about 3.5 mg per eye, about 4 mg per eye, about 4.5 mg per eye, about 5 mg per eye, about 5.5 mg per eye, about 6 mg per eye, about 6.5 mg per eye, about 7 mg per eye, about 7.5 mg per eye, about 8 mg per eye, about 8.5 mg per eye, about 9 mg per eye, about 9.5 mg per eye, or about 10 mg per eye, at a frequency of about every 8 weeks.

In some embodiments, administration of the CFI fusion constructs is an intraocular (e.g. intravitreal) administration, at a dosage of about 0.1 mg per eye, about 0.5 mg per eye, about 1 mg per eye, about 1.5 mg per eye, about 2 mg per eye, about 2.5 mg per eye, about 3 mg per eye, about 3.5 mg per eye, about 4 mg per eye, about 4.5 mg per eye, about 5 mg per eye, about 5.5 mg per eye, about 6 mg per eye, about 6.5 mg per eye, about 7 mg per eye, about 7.5 mg per eye, about 8 mg per eye, about 8.5 mg per eye, about 9 mg per eye, about 9.5 mg per eye, or about 10 mg per eye, at a frequency of about every 9 weeks.

In some embodiments, administration of the CFI fusion constructs is an intraocular (e.g. intravitreal) administration, at a dosage of about 0.1 mg per eye, about 0.5 mg per eye, about 1 mg per eye, about 1.5 mg per eye, about 2 mg per eye, about 2.5 mg per eye, about 3 mg per eye, about 3.5 mg per eye, about 4 mg per eye, about 4.5 mg per eye, about 5 mg per eye, about 5.5 mg per eye, about 6 mg per eye, about 6.5 mg per eye, about 7 mg per eye, about 7.5 mg per eye, about 8 mg per eye, about 8.5 mg per eye, about 9 mg per eye, about 9.5 mg per eye, or about 10 mg per eye, at a frequency of about every 10 weeks.

In some embodiments, administration of the CFI fusion constructs is an intraocular (e.g. intravitreal) administration, at a dosage of about 0.1 mg per eye, about 0.5 mg per eye, about 1 mg per eye, about 1.5 mg per eye, about 2 mg per eye, about 2.5 mg per eye, about 3 mg per eye, about 3.5 mg per eye, about 4 mg per eye, about 4.5 mg per eye, about 5 mg per eye, about 5.5 mg per eye, about 6 mg per eye, about 6.5 mg per eye, about 7 mg per eye, about 7.5 mg per eye, about 8 mg per eye, about 8.5 mg per eye, about 9 mg per eye, about 9.5 mg per eye, or about 10 mg per eye, at a frequency of about every 11 weeks.

In some embodiments, administration of the CFI fusion constructs is an intraocular (e.g. intravitreal) administration, at a dosage of about 0.1 mg per eye, about 0.5 mg per eye, about 1 mg per eye, about 1.5 mg per eye, about 2 mg per eye, about 2.5 mg per eye, about 3 mg per eye, about 3.5 mg per eye, about 4 mg per eye, about 4.5 mg per eye, about 5 mg per eye, about 5.5 mg per eye, about 6 mg per eye, about 6.5 mg per eye, about 7 mg per eye, about 7.5 mg per eye, about 8 mg per eye, about 8.5 mg per eye, about 9 mg per eye, about 9.5 mg per eye, or about 10 mg per eye, at a frequency of about every 12 weeks.

In In some embodiments, administration of the CFI fusion constructs is an intraocular (e.g. intravitreal) administration, at a dosage of about 0.1 mg per eye, about 0.5 mg per eye, about 1 mg per eye, about 1.5 mg per eye, about 2 mg per eye, about 2.5 mg per eye, about 3 mg per eye, about 3.5 mg per eye, about 4 mg per eye, about 4.5 mg per eye, about 5 mg per eye, about 5.5 mg per eye, about 6 mg per eye, about 6.5 mg per eye, about 7 mg per eye, about 7.5 mg per eye, about 8 mg per eye, about 8.5 mg per eye, about 9 mg per eye, about 9.5 mg per eye, or about 10 mg per eye, at a frequency of about every 13 weeks.

In some embodiments, administration of the CFI fusion constructs is an intraocular (e.g. intravitreal) administration, at a dosage of about 0.1 mg per eye, about 0.5 mg per eye, about 1 mg per eye, about 1.5 mg per eye, about 2 mg per eye, about 2.5 mg per eye, about 3 mg per eye, about 3.5 mg per eye, about 4 mg per eye, about 4.5 mg per eye, about 5 mg per eye, about 5.5 mg per eye, about 6 mg per eye, about 6.5 mg per eye, about 7 mg per eye, about 7.5 mg per eye, about 8 mg per eye, about 8.5 mg per eye, about 9 mg per eye, about 9.5 mg per eye, or about 10 mg per eye, at a frequency of about every 14 weeks.

In some embodiments, administration of the CFI fusion constructs is an intraocular (e.g. intravitreal) administration, at a dosage of about 0.1 mg per eye, about 0.5 mg per eye, about 1 mg per eye, about 1.5 mg per eye, about 2 mg per eye, about 2.5 mg per eye, about 3 mg per eye, about 3.5 mg per eye, about 4 mg per eye, about 4.5 mg per eye, about 5 mg per eye, about 5.5 mg per eye, about 6 mg per eye, about 6.5 mg per eye, about 7 mg per eye, about 7.5 mg per eye, about 8 mg per eye, about 8.5 mg per eye, about 9 mg per eye, about 9.5 mg per eye, or about 10 mg per eye, at a frequency of about every 15 weeks.

In some embodiments, administration of the CFI fusion constructs is an intraocular (e.g. intravitreal) administration, at a dosage of about 0.1 mg per eye, about 0.5 mg per eye, about 1 mg per eye, about 1.5 mg per eye, about 2 mg per eye, about 2.5 mg per eye, about 3 mg per eye, about 3.5 mg per eye, about 4 mg per eye, about 4.5 mg per eye, about 5 mg per eye, about 5.5 mg per eye, about 6 mg per eye, about 6.5 mg per eye, about 7 mg per eye, about 7.5 mg per eye, about 8 mg per eye, about 8.5 mg per eye, about 9 mg per eye, about 9.5 mg per eye, or about 10 mg per eye, at a frequency of about every 16 weeks.

In some embodiments, administration of the CFI fusion constructs is an intraocular (e.g. intravitreal) administration, at a dosage of about 0.1 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In some embodiments, administration of the CFI fusion constructs is an intraocular (e.g. intravitreal) administration, at a dosage of about 0.5 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In some embodiments, administration of the CFI fusion constructs is an intraocular (e.g. intravitreal) administration, at a dosage of about 1.0 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In In some embodiments, administration of the CFI fusion constructs is an intraocular (e.g. intravitreal) administration, at a dosage of about 1.5 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In some embodiments, administration of the CFI fusion constructs is an intraocular (e.g. intravitreal) administration, at a dosage of about 2 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In some embodiments, administration of the CFI fusion constructs is an intraocular (e.g. intravitreal) administration, at a dosage of about 2.5 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In some embodiments, administration of the CFI fusion constructs is an intraocular (e.g. intravitreal) administration, at a dosage of about 3 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In some embodiments, administration of the CFI fusion constructs is an intraocular (e.g. intravitreal) administration, at a dosage of about 3.5 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In some embodiments, administration of the CFI fusion constructs is an intraocular (e.g. intravitreal) administration, at a dosage of about 4 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In some embodiments, administration of the CFI fusion constructs is an intraocular (e.g. intravitreal) administration, at a dosage of about 4.5 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In some embodiments, administration of the CFI fusion constructs is an intraocular (e.g. intravitreal) administration, at a dosage of about 5 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In some embodiments, administration of the CFI fusion constructs is an intraocular (e.g. intravitreal) administration, at a dosage of about 5.5 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In some embodiments, administration of the CFI fusion constructs is an intraocular (e.g. intravitreal) administration, at a dosage of about 6 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In some embodiments, administration of the CFI fusion constructs is an intraocular (e.g. intravitreal) administration, at a dosage of about 6.5 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In some embodiments, administration of the CFI fusion constructs is an intraocular (e.g. intravitreal) administration, at a dosage of about 7 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In some embodiments, administration of the CFI fusion constructs is an intraocular (e.g. intravitreal) administration, at a dosage of about 7.5 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In some embodiments, administration of the CFI fusion constructs is an intraocular (e.g. intravitreal) administration, at a dosage of about 8 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In some embodiments, administration of the CFI fusion constructs is an intraocular (e.g. intravitreal) administration, at a dosage of about 8.5 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In some embodiments, administration of the CFI fusion constructs is an intraocular (e.g. intravitreal) administration, at a dosage of about 9 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In some embodiments, administration of the CFI fusion constructs is an intraocular (e.g. intravitreal) administration, at a dosage of about 9.5 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

In some embodiments, administration of the CFI fusion constructs is an intraocular (e.g. intravitreal) administration, at a dosage of about 10 mg per eye, at a frequency of about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks.

### F. Formulations

Pharmaceutical compositions containing a CFI fusion constructs of the disclosure can be formulated in any conventional manner, suitable for ocular administration, by mixing a selected amount of the polypeptide with one or more physiologically acceptable carriers or excipients, for use in the treatments provided herein. Selection of the carrier or excipient is within the skill of the administering profession and can depend upon a number of parameters. These include, for example, the mode of administration and disorder treated. The pharmaceutical compositions provided herein can be formulated for single dosage (direct) administration or for dilution or other modification. The concentrations of the compounds in the formulations are effective for delivery of an amount, upon administration, that is effective for the intended treatment. Typically, the compositions are formulated for single dosage administration, but not necessarily.

In some exemplary embodiments, the fusion construct can be concentrated to at least 150 mg/mL. In some embodiments, the fusion construct can be dosed to at least 7.5 mg in 50 uL. In some embodiments, the fusion construct can be dosed to at least 7.5 mg in about 25 ul to about 150 ul, about 30 ul to about 150 ul, about 35 ul to about 150 ul, about 40 ul to about 150 ul, about 45 ul to about 150 ul, about 50 ul to about 150 ul, about 55 ul to about 150 ul, about 60 ul to about 150 ul, about 65 ul to about 150 ul, about 70 ul to about 150 ul, about 75 ul to about 150 ul, about 80 ul to about 150 ul, about 85 ul to about 150 ul, about 90 ul to about 150 ul, about 95 ul to about 150 ul, about 100 ul to about 150 ul, about 105 ul to about 150 ul, about 110 ul to about 150 ul, about 115 ul to about 150 ul, about 120 ul to about 150 ul, about 125 ul to about 150 ul, about 130 ul to about 150 ul, about 135 ul to about 150 ul, about 140 ul to about 150 ul, about 145 ul to about 150 ul, or about 25 ul to about 150 ul.

In some exemplary embodiments, the fusion construct can be dosed to at least 7.5 mg in 25 uL, at least 7.5 mg in 30 uL, at least 7.5 mg in 35 uL, at least 7.5 mg in 40 uL, at least 7.5 mg in 45 uL, at least 7.5 mg in 50 uL, at least 7.5 mg in 55 uL, at least 7.5 mg in 60 uL, at least 7.5 mg in 65 uL, at least 7.5 mg in 70 uL, at least 7.5 mg in 75 uL, at least 7.5 mg in 80 uL, at least 7.5 mg in 85 uL, at least 7.5 mg in 90 uL, at least 7.5 mg in 95 uL, at least 7.5 mg in 100 uL, at least 7.5 mg in 105 uL, at least 7.5 mg in 110 uL, at least 7.5 mg in 115 uL, at least 7.5 mg in 120 uL, at least 7.5 mg in 125 uL, at least 7.5 mg in 130 uL, at least 7.5 mg in 135 uL, at least 7.5 mg in 140 uL, at least 7.5 mg in 145 uL, or at least 7.5 mg in 150 uL.

In some exemplary embodiments, the fusion construct is dosed to about 5 mg to 10 mg in 25 uL, 30 uL, 35 uL, 40 uL, 45 uL, 50 uL, 55 uL, 60 uL, 65 uL, 70 uL, 75 uL, 80 uL, 85 uL, 90 uL, 95 uL, 100 uL, 105 uL, 110 uL, 115 uL, 120 uL, 125 uL, 130 uL, 135 uL, 140 uL, 145 uL, or 150 uL.

### G. Pharmaceutical Compositions

The disclosure also provides pharmaceutical compositions comprising any one of the CFI fusion constructs disclosed herein, and optionally a pharmaceutical acceptable excipient or carrier, suitable for ocular administration. In some embodiments, the pharmaceutical composition is sterile. The pharmaceutical compositions may be formulated to be compatible with their intended routes of administration. In some embodiments, the pharmaceutical compositions of the disclosure are suitable for administration to a human subject, or other non-human primate. In exemplary embodiments, the pharmaceutical composition is formulated for intravitreal administration.

### H. Kits and Articles of Manufacture for Therapeutic CFI Fusion Constructs

The disclosure also provides a kit or article of manufacture comprising any one of the CFI fusion constructs disclosed herein, or any pharmaceutical composition disclosed herein. In some embodiments, the kits may further include instructional materials for carrying out any of the dosing and intraocular administration methods disclosed herein. In some embodiments, the kits may further include sterile containers or vials for holding the fusion constructs and/or pharmaceutical compositions disclosed herein. In some embodiments, the kits may further include sterile delivery devices for administering the fusion constructs and/or pharmaceutical compositions disclosed herein. In some embodiments, an article of manufacture comprises any pharmaceutical composition of the disclosure.

### EXAMPLES

### Example 1: CFI-HSA Expression, Purification, Activation, and In Vitro Sialylation

### Overview

For Example 1, reference to CFI-HSA refers to human serum albumin fused to the N-terminal end of wild type CFI (SEQ ID NO: 21).

A wild type CFI-HSA protein was expressed in Chinese hamster ovary (CHO) cells, purified with anti-albumin affinity purification, activated with furin, and purified by sizing columns. The activated CFI-HSA protein was subjected to in vitro sialylation to increase the total sialylation of CFI-HSA. Finally, the sialylated protein was purified using anti-albumin affinity purification and polished by size-exclusion column chromatography.

### Expression

The CFI-HSA gene (SEQ ID NO: 21) was synthesized (ThermoFisher Scientific, Geneart, Regensburg, Germany), with the human serum albumin at the amino terminus of the CFI protein. The protein was made with the signal sequence of SEQ ID NO: 2, which was removed during expression. The amino terminal albumin tag was connected to the CFI gene through a linker (SEQ ID NO: 6). The gene of CFI-HSA was inserted into an expression vector (Lake Pharma, Hayward, CA) utilizing standard molecular biology techniques. The resulting plasmid DNA was transformed into E. coli. The transfected E. coli were grown in 200 ml of LB media for expression of plasmid DNA and harvested utilizing standard techniques. The plasmid DNA was run on an agarose gel for quality assessment and sequence confirmed before proceeding to transfection.

1.0 liter of suspension TunaCHO™ cells were seeded in a shake flask and were expanded using serum-free chemically defined medium. On the day of transfection, the expanded cells were seeded into a new flask with fresh medium. The plasmid DNA was transiently transfected into the CHO cells using Lipofectamine 2000 (ThermoFisher Scientific). The cells were maintained as a batch-fed culture until the end of the production run. The protein was expressed for 14 days at 37° C. at 125 RMP with 8% CO₂ concentration. Cells were centrifuged and supernatant was collected for purification of secreted CFI-HSA at the end of 14 days expression.

### Purification

The supernatant with expressed CFI-HSA protein was passed through a 10 ml gravity flow column of CaptureSelect™ human albumin affinity matrix (ThermoFisher Scientific). Column-bound protein was washed with 10 column volume of 20 mM sodium phosphate buffer. Bound CFI-HSA protein was eluted in two steps: first, with 3 column volume of 20 mM Tris-HCl, pH 7.0 buffer with and 2 M MgCl₂, and second, with 3 column volume of 20 mM citric acid, pH 3.0. Elution from both steps 1 and 2 was collected in 5 ml fractions. Each fraction of the step 2 elution was neutralized with 10% of neutralization buffer (1.5 M tris-HCL pH 7.4). All fractions were analyzed by reducing and non-reducing SDS-PAGE electrophoresis and bands were visualized by SimplyBlue™ SafeStain (ThermoFisher Scientific). CFI-HSA runs as a 130 kDa band on a non-reducing gel and as 102 kDa and 28 kDa bands on a reducing gel. Fractions with maximum CFI-HSA concentration and purity were pooled for further processing.

### Furin Activation

Cleavage of CFI-HSA for producing the protein in its activated form was performed by incubation of 4 μg of recombinant furin per mg of purified CFI-HSA in Tris-NaCl (tris buffered saline), 2.5 mM CaCl₂) and 0.5% CHAPS at 30° C. for 18 hours. The CFI-HSA protein concentration was maintained at 1.4 mg/ml. This results in more than 90% activation of the protein. The activated protein was separated from inactivated CFI-HSA, and other proteins by size-exclusion chromatography. Size exclusion chromatography (SEC) was performed using a HiLoad 16/600 Superdex 200 column (GE Healthcare Life Sciences) and phosphate buffer saline (PBS, 137 mM NaCl, 2.7 mM KCl, 10 mM Na₂HPO₄, 2 mM KH₂PO₄, pH 7.4) as the mobile phase. Collected fractions were analyzed by CE-SDS (LabChip GXII, Perkin Elmer). Fractions containing the target protein were pooled and analyzed by SE-UPLC.

### In Vitro Sialylation

The activated CFI-HSA protein was subject to in vitro sialylation. Briefly, the sialylation was carried out in a two-step enzymatic reaction. First, a galactosylation reaction of CFI-HSA was performed in a 200 μl volume utilizing a 1:200 molar ratio of galactosyltransferase (GalT1) enzyme and CFI-HSA in 10 mM UDP-Galactose, 5 mM MnCl₂, and 100 mM MES, pH 6.5 buffer. Galactosylated CFI-HSA was purified from the reaction mixture by CaptureSelect™ Human Albumin affinity chromatography, as described earlier. Next, the sialylation reaction was performed in a 250 μl volume utilizing a 1:50 molar ratio of enzyme alpha 2,6-sialyl transferase and purified CFI-HSA in 80 μM Alkaline phospahatase, 6.1 mM CMP-NANA, 10 mM ZnCl₂ and 200 mM MES buffer, pH 6.5 at 37° C. for 1 hour. The sialylated CFI-HSA protein was purified from the reaction mixture by CaptureSelect™ Human Albumin affinity chromatography. The extent and characteristics of the sialic acid chain on CFI-HSA was determined by utilizing an Agilent/Prozyme Analytical service, GS-SAP method for total sialic acid quantitation (Agilent GS48), and mass spectrophotometric (MS) analysis (Lake Pharma analytical service), described in further detail below.

Briefly, total sialic acid quantitation was performed by mixing 20 μl of each sample with 10 μl of release reagent in a 96 well plate. The reaction mixture was incubated for 2 hours at 80° C. The samples were cooled to room temperature and 10 μl of labeling reagent was added to each sample for a further incubation of 3 hours at 50° C. The samples were again cooled down to room temperature and 160 μl of de-ionized (dI) water was added to bring the total volume to 200 μl. 10 μl of sample was injected in the Agilent UHPLC Poroshell C18 column to run at a flow rate of 0.4 ml/minute at 30° C. in 4% methanol, 8% acetonitrile in water (Line A1) and 100% ACN (Line B1). The peaks were recorded at 373/448 nm wavelength. A standard curve of total peak area versus picomoles (pmol) of sialic acid was generated by running 1-2000 pmol of NANA (N-acetylneuraminic acid, Neu5Ac) supplied with the kit on the same column. Total sialic acid of each sample was quantitated by comparing the peak area of samples against the standard curve. The sialylation obtained is summarized in Table 1.1 below.

TABLE 1.1
Sialylation Assay Results

| | Sialic Acid (Neu5Ac) |
|---|---|
| Protein | pmol/ug protein |
| Recombinant CFI-HSA | 35 ± 0.7 |
| Recombinant CFI-HSA-In | 69 ± 2.2 |
| vitro sialylated | |
| Bovine Fetuin control | 222 ± 2.6 |

The mass spectrometric analysis was performed by a standard trypsin Q-TOF mass spectrometer. Briefly, all samples were treated, reduced and alkylated by DTT and iodoacetamide, followed by trypsin digestion. The digested samples were analyzed by Waters ACQUITY UPLC coupled to a Xevo G2-XS-QTOF mass spectrometer using a protein BEH C18 column. The performed analysis is summarized in Table 1.2 below.

TABLE 1.2
Peptide Analysis Results

| | | Recombinant | Recombinant |
|---|---|---|---|
| | | CFI-HSA | CFI-HSA- |
| | Glycans | (%) | In vitro sialylated (%) |
| Peptide | Man5 | 7.1 | 16.24 |
| Analysis | G0 | 2.39 | 0 |
| | G0F | 0.88 | 0 |
| | G1 | 6.07 | 0 |
| | G1F | 6.68 | 0.03 |
| | G2 | 13.62 | 2.37 |
| | G2F | 24.81 | 1.78 |
| | G2FSA | 13.38 | 19.44 |
| | G2FSA2 | 21.48 | 53.91 |

### Polishing

Purified CFI-HSA protein was subjected to size-exclusion chromatography (SEC) using a HiLoad 16/600 Superdex 200 column (GE Healthcare Life Sciences) and phosphate buffer saline as the mobile phase. Collected fractions were analyzed by CE-SDS (LabChip GXII, Perkin Elmer). Fractions containing the target protein were pooled, and the concentration was brought to 5 mg/ml, and the samples were flash frozen for storage at −80° C.

### N-Terminal Albumin Fusion Provides Solubility and Facilitates Activation of CFI-HSA

Activation was compared between CFI-HSA and wild-type CFI without an albumin, or other fusion tag (WT-CFI). A gene construct for WT-CFI was expressed essentially as described above for CFI-HSA. The recombinant WT-CFI protein showed moderate purity by reduced SDS-PAGE, however, significant High Molecular Weight Species (HMWS) and aggregates under reduced and non-reduced conditions were observed. For CFI-HSA, which has the addition of an N-terminal HSA tag, transient expression using the TunaCHO™ cells followed by purification as described in above showed no HMWS or aggregates on reduced and non-reduced SDS-PAGE. Activation of the purified recombinant CFI with furin resulted in a further increase in aggregates and HMWS with almost complete polydispersity. Furthermore, essentially no activated CFI was observed by reducing SDS-PAGE. On the contrary, the addition of furin efficiently activated CFI-HSA almost completely under the same conditions and the CFI-HSA protein remained as a monomer under non-reduced conditions with no evidence of aggregates and HMWS. When compared to CFI lacking any fusion tags, there is a significant and unexpected benefit of the N-terminal HSA tag for maintaining solubility, monodispersity and efficient furin activation.

### Example 2: CFI-HSA Half-Life in the Vitreous Humor of Non-Human Primates

For Example 2, reference to CFI-HSA refers to human serum albumin fused to the N-terminal end of wild type CFI (SEQ ID NO: 21).

### Pharmacokinetics and Pharmacodynamics of CFI-HSA Following Intravitreal Injection

The ocular pharmacokinetics of the N-terminal albumin fusion of wild-type CFI-HSA were examined after intravitreal dosing to six AGMs. The six animals were divided into two groups treated at 2 dose levels: one group received a single intravitreal injection of 500 μg of CFI-HSA (right eye, OD, N=3) and the other group received a single intravitreal injection of 250 μg of CFI-HSA (right eye, OD, N=3). The left eye (OS) of all six animals was injected with an equivalent volume of 100 μL of sterile PBS for injection as a vehicle control. Non-terminal, vitreous humor samples (100 μL) were taken on days 1, 7, 14, 21 and 28 post dosing. Vitreous humor CFI-HSA drug concentrations were determined using a quantitative electrochemiluminescence (ECL) antigen assay optimized for measuring CFI-HSA in vitreous humor of AGMs. The assay employs coating of anti-CFI antibody (clone OX21, LS Bio, Seattle WA) at 2 μg/ml on the Meso Scale Discovery (MSD, Rockville, MA) assay plate to capture the CFI-HSA levels. Detection of the captured CFI-HSA is performed with a goat polyclonal anti-HSA antibody (Abcam, Cambridge, MA) at 0.5 μg/ml conjugated with SULFO-TAG which emits light [electrochemiluminescence (ECL)] on application of an electric potential. The ECL relative light units (RLU) is measured on a MESO® SECTOR S 600 Reader and the unknown CFI-HSA concentrations in vitreous humor are interpolated from a standard curve ranging from 0.05 μg/ml to 40 μg/ml Factor I-HSA. Data are provided in Table 2.1.

TABLE 2.1
CFI-HSA levels at day 1, 7, 14, 21
and 28 after intravitreal dosing

| Dose | No of | | Levels of Factor I- |
|---|---|---|---|
| level | animals | timepoints | HSA +/− SD (μg/ml) |
| 5.01 | 3 | baseline | BLQ |
| mg/ml | | Day 1 | 66.4 +/− 18.9 |
| | | Day 7 | 34.7 +/− 5.5 |
| | | Day 14 | 9.0 +/− 6.4 |
| | | Day 21 | 2.0 +/− 1.3 |
| | | Day 28 | 0.9 +/− 0.6 |
| 2.50 | 3 | baseline | BLQ |
| mg/ml | | Day 1 | 46.9 +/− 11.1 |
| | | Day 7 | 12.5 +/− 5.0 |
| | | Day 14 | 2.8 +/− 3.0 |
| | | Day 21 | 0.2 +/− 0.1 |
| | | Day 28 | 0.6 |

*BLQ: measurement below the limit of quantification of the assay

Non-compartmental analysis yielded apparent ocular terminal half-lives of 3.6 and 4.1 days for the 250 and 500 μg dose levels, respectively.

TABLE 2.2
Estimated PK parameters for CFI-HSA in
vitreous humor of African Green Monkeys

| Parameters | 500 μg | 250 μg |
|---|---|---|
| Terminal half-life (days) | 4.1 | 3.7 |
| Tₘₐₓ (days) | 1.0 | 1.0 |
| Cₘₐₓ (μg/ml) | 66.4 | 46.9 |
| MRT (days) | 6.0 | 4.4 |
| AUC 0-inf (μg/ml × days) | 543.2 | 271.5 |
| AUC 0-t (μg/ml × days) | 537.8 | 268.5 |

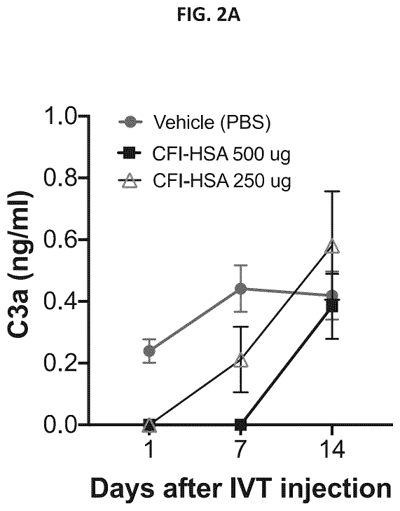

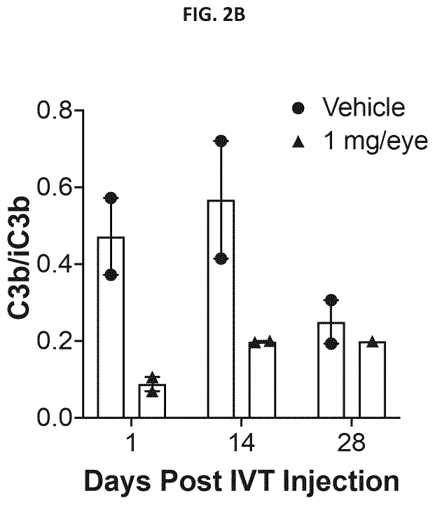

Complement Component 3a (C3a) levels in vitreous humor were determined by ELISA using the Quidel kit for C3a ELISA for the 250 μg and 500 μg doses (FIG. 2). CFI-HSA fusion construct reduces ocular C3a levels in a dose-dependent manner up to 7 days after intraocular injection. The increase in C3b degradation by CFI-HSA reduces the complex formation between C3b and Bb which leads to a reduction of C3 cleavage into C3a and C3b via the amplification loop of the alternative pathway.

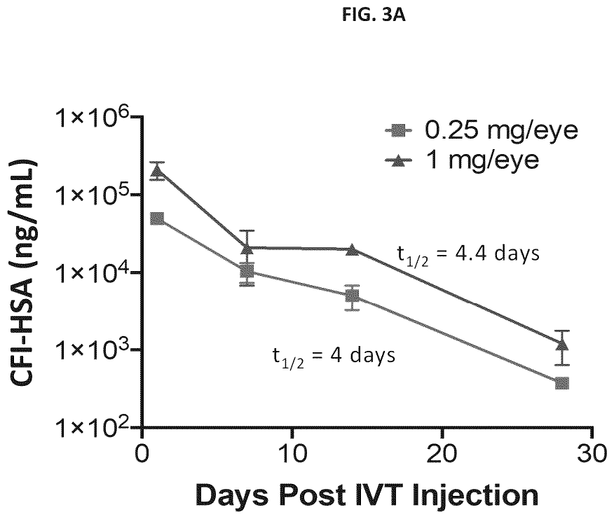

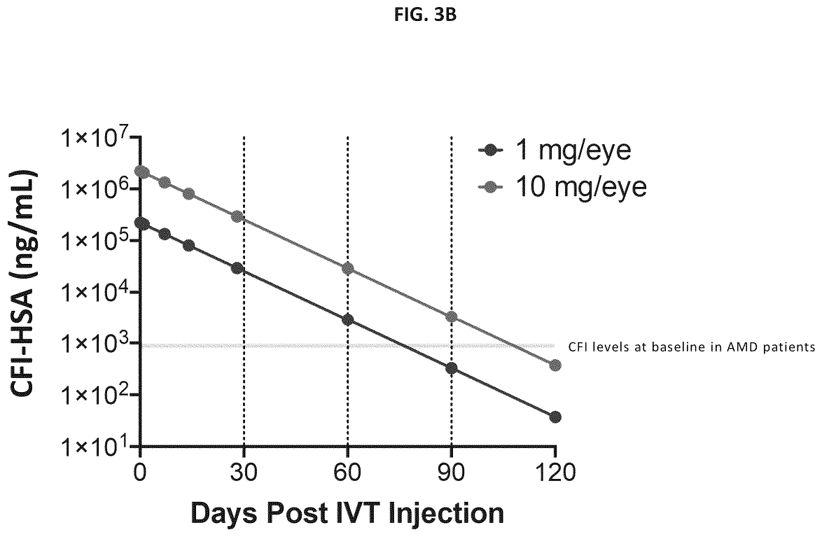

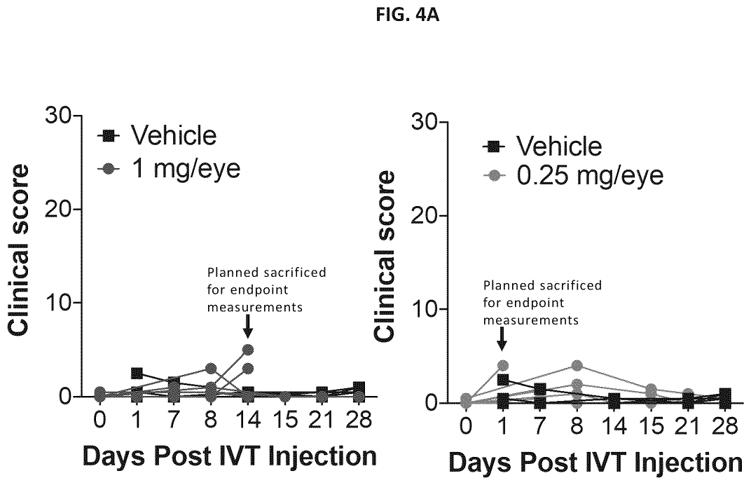

The ocular pharmacokinetics of the N-terminal albumin fusion of wild-type CFI-HSA were examined after intravitreal dosing to six AGMs. The six animals were divided into two groups treated at 2 dose levels: one group received a single intravitreal injection of 0.25 mg of CFI-HSA (OU, N=4), 1.0 mg of CFI-HSA (OU, N=4) and a control group that received a single intravitreal injection of vehicle (OU, N=4). The eyes (OU) of all 12 animals were injected with an equivalent volume of 100 μL of test article or vehicle control. Animal received ocular exams (see Example 4) on days 0, 1, 7 or 8, 14 or 15, 21 and 28 post dosing. Animals were euthanized on days 1, 7, 14 and 28 for PK-PD analyses and histopathology. The vitreous humor CFI-HSA drug concentrations were determined using a quantitative electrochemiluminescence (ECL) antigen assay optimized for measuring CFI-HSA in vitreous humor of AGMs as described above. Evaluated terminal vitreous half-life measurements were 4 days and 4.4 days for the 0.25 mg and 1.0 mg doses, respectively (FIG. 3A).

Complement Component 3b (C3b) and the cleavage fragment iC3b levels in vitreous humor were determined by chemiluminecent western blot using an anti-C3 antibody and quantification by densitometry for the 1.0 mg dose (FIG. 2B). The CFI-HSA fusion construct reduced ocular iC3b levels for up to 14 days after intraocular injection. The increase in C3b degradation by CFI-HSA is represented as a ratio of C3b/iC3b for days 1, 7 and 14, showing a significant increase in cleavage in eyes receiveing a 1.0 mg injection of CFI-HSA compared to vehicle controls (FIG. 2B).

### Example 3: Pharmacokinetics Modeling of CFI-HSA Intravitreal Injections for Human Dosing

The half-life of CFI-HSA after intravitreal injection of CFI-HSA was measured over time in nonhuman primates. A half-life of about 4 days was found to preserve dose proportionality across 3 dose levels in AGMs, as exemplified in FIG. 3A. In addition, CFI-HSA cleavage activity was detected up to 14 days via Western blot (FIG. 2B).

Based on AGM data, a vitreous PK model for human dosing was generated, as exemplified in FIG. 3B. Without being held to theory or mechanism, intravitreal injections of CFI-HSA at 1 mg per eye is predicted to maintain baseline CFI levels above that of AMD patients over 60 days post injection. Intravitreal injections of CFI-HSA at 10 mg per eye is predicted to maintain baseline CFI levels above that of AMD patients over 90 days post injection.

### Example 4: Safety Profile of CFI-HSA Intravitreal Injections in Non-Human Primates

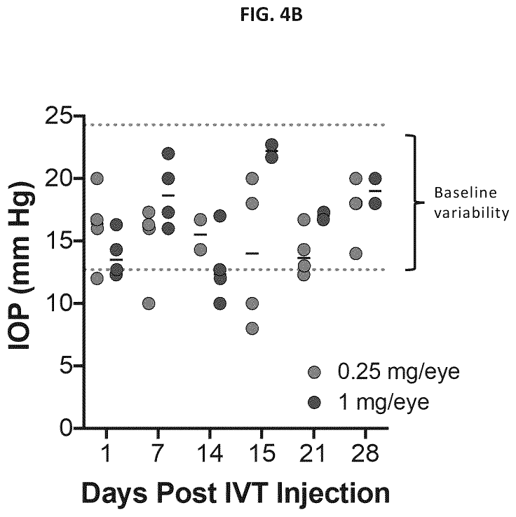

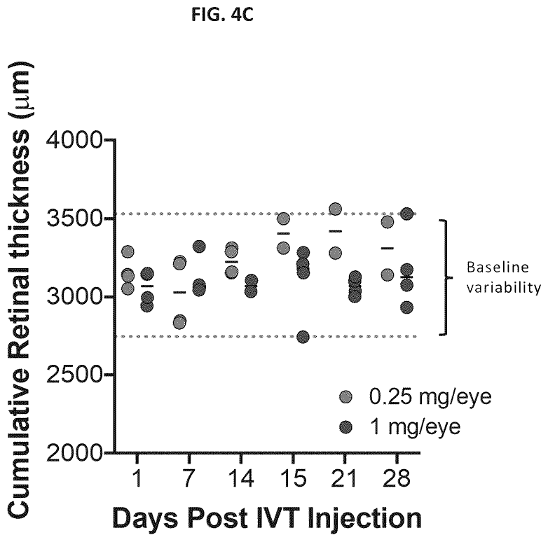

The safety profile of CFI-HSA intravitreal injections of CFI-HSA was determined in non-human primates. Post-injection, AGMs were assessed for a clinical score via a slit lamp test on days 0, 1, 7 or 8, 14 or 15, 21 and 28 or until the termination day. A clinical score using the slit lamp test was generated over time post intravitreal injection of CFI-HSA, as exemplified in FIG. 4A. AGM subjects were also assessed for intraocular pressure post-injection of CFI-HSA. The intraocular pressure of injected AGM subjects was measured over time, as exemplified in FIG. 4B. The intraocular pressure of individual injected AGM subjects was found to be within the baseline variability and thus stable. Lastly, the retinal thickness of AGM subjects was assessed post injection of CFI-HSA. The retinal thickness of injected AGM subjects were measured over time, as exemplified in FIG. 4C. The retinal thickness of individual injected AGM subjects was found to be within the baseline variability and thus stable.

1. A method of treating or preventing an ocular condition in a human subject in need thereof, comprising:
administering to the subject a fusion construct comprising a
(a) Complement Factor I (CFI) comprising the amino acid sequence of SEQ ID NO. 5, or a variant thereof, and
(b) a binding partner,
wherein the route of administration is intraocular, and wherein the fusion construct is administered at a:
(i) dose of about 0.1 to about 10 mg per eye; and/or
(ii) frequency of about every 1 to about 4 months.

2. The method of claim 1, wherein the variant of CFI comprises at least 70% sequence identity with the amino acid sequence of SEQ ID NO. 5.

3. The method of claim 2, wherein the variant of CFI comprises a CFI of SEQ ID NO: 5 comprising one or more of the substitutions of T377G, N422K, E457G, and N531G.

4. The method of claim 2, wherein the variant of CFI comprises a CFI of SEQ ID NO: 5 comprising (a) the substitutions of N422K, E457G, and N531G, or (b) the substitutions of T377G, E457G, and N531G.

5. (canceled)

6. The method of claim 1, wherein fusion protein comprises the Complement Factor I (CFI) comprising the amino acid sequence of SEQ ID NO. 5.

7. The method of claim 1, wherein the binding partner is a serum albumin comprising the amino acid sequence of SEQ ID NO. 7, or a variant thereof, optionally wherein the variant of serum albumin comprises at least 70% sequence identity with the amino acid sequence of SEQ ID NO. 7.

8. (canceled)

9. The method of claim 1, wherein the fusion construct comprises at least 70% sequence identity with the amino acid sequence of SEQ ID NO. 21.

10. The method of claim 1, wherein the fusion construct comprises the amino acid sequence of SEQ ID NO. 21.

11. The method of claim 1, wherein the binding partner is a cofactor of CFI.

12. The method of claim 11, wherein the binding partner comprises:
(a) a Factor H cofactor comprising the amino acid sequence of SEQ ID NO. 22 or SEQ ID NO: 23, a domain thereof, or a part of a domain thereof; or
(b) a CR1, a domain thereof, or a part of a domain thereof.

13.-15. (canceled)

16. The method of claim 1, wherein the ocular condition is dry Age-related Macular Degeneration (AMD), wet AMD, or Geographic Atrophy (GA).

17.-19. (canceled)

20. The method of claim 1, wherein the human subject carries one or more mutations to CFI selected from the group consisting of: G119R, L131R, V152M, G162D, R187Y, R187T, T203I, A240G, A258T, G287R, A300T, R317W, R339Q, V412M, P553S, K441R, R339Ter, R317Q, G261D, R187Q, and R187Ter.

21. The method of claim 1, wherein the dose is about 0.1 mg to about 10 mg per eye, about 0.5 mg to about 10 mg per eye, about 1 mg to about 10 mg per eye, about 1.5 mg to about 10 mg per eye, about 2 mg to about 10 mg per eye, about 2.5 mg to about 10 mg per eye, about 3 mg to about 10 mg per eye, about 3.5 mg to about 10 mg per eye, about 4 mg to about 10 mg per eye, about 4.5 mg to about 10 mg per eye, about 5 mg to about 10 mg per eye, about 5.5 mg to about 10 mg per eye, about 6 mg to about 10 mg per eye, about 6.5 mg to about 10 mg per eye, about 7 mg to about 10 mg per eye, about 7.5 mg to about 10 mg per eye, about 8 mg to about 10 mg per eye, about 8.5 mg to about 10 mg per eye, about 9 mg to about 10 mg per eye, or about 9.5 mg to about 10 mg per eye.

22. The method of claim 1, wherein the dose is about 0.1, 0.5, 1, 5, or 10 mg per eye.

23. The method of claim 1, wherein the frequency is about every 1, 2, 3, or 4 months.

24. (canceled)

25. The method of claim 1, wherein the fusion construct is administered to one eye.

26. The method of claim 1, wherein the fusion construct is administered to both eyes.

27.-28. (canceled)

29. The method of claim 1, wherein the subject's intraocular pressure or retinal thickness is stable after treatment.

30. (canceled)

31. The method of claim 1, wherein upon administration, the fusion construct exhibits an activity in the subject that is at least equivalent to the activity that an endogenous CFI exhibits.

32. The method of claim 1, wherein upon administration, the baseline CFI level is maintained above that of the baseline CFI level of a reference subject who did not receive the injection.


SEQUENCE LISTING (25 sequences)

SEQ ID NO: 1 - protein, 583 aa, Homo sapiens
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 2 - protein, 20 aa, Mus musculus
METDTLLLWVLLLWVPGSTG

SEQ ID NO: 3 - protein, 18 aa, Homo sapiens
MKLLHVFLLFLCFHLRFC

SEQ ID NO: 4 - skipped

SEQ ID NO: 5 - protein, 565 aa, Homo sapiens
KVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 6 - protein, 6 aa, synthetic construct
GGSSGG

SEQ ID NO: 7 - protein, 585 aa, Homo sapiens [sequence omitted]

SEQ ID NO: 8-20 - skipped (13 entries)

SEQ ID NO: 21 - protein, 1156 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGSSGGKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 22 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 23 - protein, 415 aa, Homo sapiens
MRLLAKIICLMLWAICVAEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEAGGGGGGGGGGGGGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRENLYFQGHHHHHH

SEQ ID NO: 24 - protein, 22 aa, synthetic construct
GGSSGGSSGGSSGGSSGGSSGG

SEQ ID NO: 25 - protein, 102 aa, synthetic construct
  REPEAT 1..4: note = single repeat of (GGSS)n where n=1-25
  VARIANT 1..100: note = up to 24 copies of (GGSS) can be deleted
GGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGG